(19) United States
(12) Patent Application Publication
(10) Pub. No.: US 2023/0293554 A1
(43) Pub. Date: Sep. 21, 2023

(54) ISOLATION OF CANNABINOIDS USING MESOPOROUS MATERIALS

(71) Applicant: UNIVERSITY OF YORK, York, North Yorkshire (GB)

(72) Inventors: Thomas M. ATTARD, York, North Yorkshire (GB); Jennifer ATTARD, York, North Yorkshire (GB); Vitaliy L. BUDARIN, York, North Yorkshire (GB); Alexandra LANOT, York, North Yorkshire (GB); Con Robert MCELROY, York, North Yorkshire (GB); James H. CLARK, York, North Yorkshire (GB)

(21) Appl. No.: 18/005,734

(22) PCT Filed: Jul. 22, 2021
(86) PCT No.: PCT/GB2021/051901

§ 371 Date: Jan. 17, 2023

(30) Foreign Application Priority Data
Jul. 23, 2020 (GB) 2011442.7

(51) Int. Cl. A61K 31/00; A61K 36/185

(52) U.S. Cl. CPC A61K 31/658; A61K 36/185; A61K 2236/37; A61K 2236/35; A61K 2236/53; A61K 2236/333

(57) ABSTRACT

The invention relates to a simple, cost-effective and eco-friendly process of recovering one or more cannabinoids from complex natural products, especially cannabidiol (CBD) and/or cannabidiolic acid (CBDA) from raw Cannabis plant material or extracts thereof.

### FIELD OF THE INVENTION

The invention relates to a simple, cost-effective and eco-friendly process of recovering one or more cannabinoids from complex natural products, especially cannabidiol (CBD) and/or cannabidiolic acid (CBDA) from raw Cannabis plant material or extracts thereof.

### BACKGROUND TO THE INVENTION

Cannabis sativa L. is a monospecific C3 plant with several subspecies (C. sativa, C. indica, C. ruderalis). There are over 400 chemical entities found in Cannabis including cannabinoids, a class of C₂₁ terpenophenolic compounds. There has been a substantial increase in the global demand for cannabinoids, especially with the ever-increasing legalisation of medicinal Cannabis. The medical use of Cannabis is now legal in a host of countries including UK, Germany, Italy, Netherlands, Switzerland, Portugal, Poland, Israel, US, Canada, Australia and New Zealand.

Over 60 cannabinoids have been extracted and isolated, including Δ9-tetrahydrocannabinolic acid (THCA), Δ9-tetrahydrocannabinol (THC), cannabigerol (CBG), cannabidiolic acid (CBDA), cannabidiol (CBD), cannabichromene (CBC) and cannabidivarin (CBDV). CBD has been the main cannabinoid investigated for medical use due to its non-psychoactive properties as well as its large abundance in the acidic form (CBDA). CBD has been shown to have a plethora of pharmacological properties in the treatment of neurological and central nervous system (CNS) disorders, consequently possessing significant therapeutic importance. The medicinal advances for use of CBD have seen investigations in seizures, spasms, migraines, pain relief, anxiety, glaucoma, anti-nausea, anti-bacterial and anti-inflammatory purposes.

While the extraction of cannabinoids is relatively straightforward, the conventional purification of cannabinoids is a long exhaustive process as the cannabinoids have to be separated from a crude extraction mixture comprising several different families of hydrophobic compounds, including but not limited to long-chain saturated and unsaturated fatty acids, fatty alcohols, fatty aldehydes, hydrocarbons, wax esters, sterols, terpenes etc.

The conventional extraction and purification of cannabinoids is a stepwise process, comprising: (i) extraction, (ii) winterisation, (iii) chlorophyll removal, (iv) decarboxylation and (v) further purification and fractionation—each step increasing the purity of the cannabinoid content. This protracted process is both time-consuming and costly.

It is an aim of certain embodiments of the present invention to at least partially mitigate the problems associated with the prior art.

It is an aim of certain embodiments of the present invention to provide improved methods for obtaining cannabinoids from natural products such as complex natural extracts.

### SUMMARY OF CERTAIN EMBODIMENTS OF THE INVENTION

The invention relates to a method of obtaining one or more cannabinoids (both acid and free form) from extracted or non-extracted plant material comprising a host of different compounds. The method of the invention avoids the need for a number of time-consuming and energy intensive steps associated with conventional methods requiring, for example, winterisation, distillation and/or chromatography.

Advantageously, the invention allows the isolation of one or more cannabinoids (including their acids) at a high yield, high degree of purity and substantially free of ballast (e.g. waxes, sterols and other lipid-soluble components).

In certain embodiments, the one or more cannabinoids are substantially free of volatile terpenes.

In certain embodiments, the one or more cannabinoids are recovered at approximately the same ratio as present within the natural product used as starting material. Thus, the method of the invention may lead to no fractionation of the cannabinoids.

In certain embodiments, the one or more cannabinoids are at a sufficient purity to be processed directly into pharmaceutical dosage forms (e.g. tablets, capsules, sprays, liquid formulations and the like).

Advantageously, the invention allows extraction and purification steps to be performed within the same “one-pot” system using environmentally friendly compounds and materials. The claimed method therefore allows non-extracted plant material to be used as starting material.

Advantageously, the invention does not require high temperatures leading to decarboxylation (e.g. temperatures of 100° C. or more). Thus, the invention allows the isolation of cannabinoids in their acid form.

Advantageously, the method of the invention utilizes solid phase extraction, may be performed quickly and allows commercial scale.

Accordingly, certain aspects of the present invention provide inter alia a method of isolating one or more cannabinoids from a natural product by solid phase extraction, wherein the method comprises the following steps:
(a) contacting the natural product with a primary solvent to obtain a feed solution (e.g., liquid phase);
(b) passing the feed solution through a mesoporous material, thereby adsorbing the one or more cannabinoids on to the mesoporous material (e.g., solid phase);
(c) passing a wash solution through the mesoporous material; and
(d) passing a second solvent through the mesoporous material, thereby desorbing the one or more cannabinoids from the mesoporous material into an elution solution.

Aptly, the primary solvent is non-polar and the second solvent is polar.

Aptly, the surface of the mesoporous material contains aromatic and/or oxygenated functionality.

Aptly, the natural product is derived from Cannabis and/or the one or more cannabinoids comprise CBD and/or CBDA.

The invention further provides a method of making a pharmaceutical composition comprising one or more cannabinoids as an active ingredient, wherein the method comprises:
(i) isolating one or more cannabinoids according to any method as described herein; and
(ii) formulating the isolated cannabinoid(s) with one or more pharmaceutically acceptable diluents, carriers or excipients.

The invention further provides use of any mesoporous material as described herein in isolating one or more cannabinoids from a natural product.

The invention further provides a cannabinoid-rich extract obtained by any method as described herein.

### BRIEF DESCRIPTION OF DRAWINGS

Certain embodiments of the present invention will now be described hereinafter, by way of example only, with reference to the accompanying drawings in which:

FIG. 1 illustrates the set-up of the auto sampler for runs of adsorption and desorption experiments.

FIG. 2 illustrates a schematic representation of the SFE-500 extractor.

FIG. 3 illustrates a modified one pot rig.

FIG. 4 illustrates a schematic of the tunability of Starbon properties and chemistries.

FIG. 5 illustrates a GC chromatograph of hemp tops (HTs): A) Crude hemp extract, B) Adsorption phase, C) Desorption phase.

FIG. 6 is a graph illustrating the A-series % CBD recovery quantified by external standard.

FIG. 7 is a graph illustrating the difference in cannabinoid (A) or CBD (B) recovery from HTs by using normal method (blue, left hand bars) versus implementation of hexane rinse and washing steps (green, right hand bars).

FIG. 8 is a graph illustrating the CBD recovery across all Starbon® materials tested using hexane/methanol (1.25 mg/mL HT extract concentration).

FIG. 9 is a graph illustrating the recovery of CBD from various solvents quantified by external standard.

FIG. 10 is a graph illustrating the desorption of HTs using hexane and various desorption solvents on A300.

FIG. 11 illustrates GC chromatograms for desorption of HTs using hexane as the adsorption solvent and (i) Methanol (ii) Ethanol.

FIG. 12 illustrates GC-FID chromatograms of (i) Crude hemp top extract (ii) Desorption phase. Cannabinoid isolation by A300 is shown in Desorption phase.

FIG. 13 is a graph illustrating the recovery of CBD from HT extract at different concentrations using hexane/ethanol.

FIG. 14 is a graph illustrating the recovery of cannabinoids from HT extract at different concentrations using hexane/ethanol.

FIG. 15 is a graph illustrating the loading and desorption of CBD for P300 Starbons at increasing concentrations using hexane/ethanol.

FIG. 16 is a graph illustrating the Pecbons recovery of CBD from HTs at different concentrations using hexane/ethanol.

FIG. 17 is a graph illustrating the large-scale set up of HT extraction using hexane/ethanol and A300.

FIG. 18 is a graph illustrating the 1H NMR spectrum for desorption from large-scale Algibon extraction.

FIG. 19 illustrates NMR labels and assignments for CBDA.

FIG. 20 illustrates 1H NMR spectra of CBD and CBDA in CDCl₃.

FIG. 21 is a graph illustrating the 13C NMR spectrum of CBDA from desorption of large-scale A300 extraction.

FIG. 22 is a graph illustrating the CBD recovery of large-scale P300 extractions using hexane/ethanol on multiple adsorption/desorption runs.

FIG. 23 illustrates GC-FID chromatograms representing 3 extracts. From top to bottom; Original hemp waste extract, adsorption, desorption phases. Fa—fatty acids, Ak—alkanes, Ac—Alcohols.

FIG. 24 illustrates images of; 1) fraction that did not adsorb on the A300, and 2) fractions desorbed with ethanol.

FIG. 25 is a graph illustrating the GC-EI-MS spectra of one pot hemp dust desorption phase.

### DETAILED DESCRIPTION

Further features of certain embodiments of the present invention are described below. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Units, prefixes and symbols are denoted in their Système International de Unitese (SI) accepted form. Numeric ranges are inclusive of the numbers defining the range.

Methods of Isolating Cannabinoids from a Natural Product

The invention provides a method of isolating one or more cannabinoids from a natural product by solid phase extraction (SPE). Typically, the method of the invention allows recovery and/or purification of one or more cannabinoids. For example, the elution solution recovered in the method of the invention may retain one or more cannabinoids but contain no (or trace amounts only) of non-desirable compounds such as long chain saturated and unsaturated fatty acids, fatty alcohols, aldehydes, hydrocarbons, wax esters, sterols or terpenes.

The method of the invention typically comprises at least the following steps:
(a) contacting the natural product with a primary solvent to obtain a feed solution (e.g., liquid phase);
(b) passing the feed solution through a mesoporous material, thereby adsorbing the one or more cannabinoids to the mesoporous material (e.g., solid phase);
(c) optionally passing a wash solution through the mesoporous material; and
(d) passing a second solvent through the mesoporous material, thereby desorbing the one or more cannabinoids from the mesoporous material into an elution solution, thereby isolating the one or more cannabinoids.

As used herein, “solid phase extraction” (or “SPE”) refers to an extraction method in which the one or more cannabinoids dissolved or suspended within the feed solution (e.g., liquid phase) are separated from other compounds within the mixture according to their physical and/or chemical properties. This is achieved by passing the feed solution through the mesoporous material (e.g., stationary phase). The one or more cannabinoids of interest are adsorbed (e.g., retained) on the mesoporous material by strong (but reversible) interactions. Typically, the portion of the feed solution that passes through the mesoporous material is discarded. The portion retained on the mesoporous material is optionally washed and removed for collection in an additional step in which the mesoporous material is rinsed with an appropriate eluent.

The method of the invention focuses on highly selective binding of the one or more cannabinoids to the mesoporous material. The flow of the feed solution through the mesoporous material is noncontinuous and the cannabinoids are eluted in a separate discrete step. Advantageously, the claimed method therefore differs from other techniques such as reverse phase chromatography or the like which require a continuous mobile phase and the continuous collection of flow through.

As used herein, the term “cannabinoid” encompasses any cannabinoid that may be present in the natural product, including, for example, cannabigerols (CBG), cannabichromenes (CBC), cannabidiol (CBD), tetrahydrocannabionol (THC), cannabinol (CBN), cannabinodiol (CBL), cannabicyclol (CBL), cannabielsoin (CBE), cannabitriol (CBT) and the like. In preferred embodiments, the invention provides a method of isolating one or more cannabidiols (CBDs) from the natural product.

Advantageously, the claimed method may be performed under conditions that do not lead to decarboxylation. For example, the invention may be performed at temperatures less than about 100° C., less than about 80° C., less than about 60° C., or less than about 50° C. The invention does not require high temperatures leading to decarboxylation (e.g. temperatures of 100° C. or more). Thus, the invention allows the isolation of cannabinoids in their acid form.

As used herein, the term “cannabinoid” also encompasses any cannabinoid acids that may be present in the natural product (i.e. any naturally occurring cannabinoid acids of the free cannabinoids as described above). In preferred embodiments, the invention provides a method of isolating one or more cannabidiolic acids (CBDAs) from the natural product.

In certain embodiments, the method of the invention further comprises a decarboxylation step. For example, in certain embodiments the method further comprises a heating step of about 100° C. to about 150° C. to convert the cannabinoid acids to free cannabinoids.

As used herein, the term “natural product” (or “complex natural product”) includes any biological material that contains one or more cannabinoids. Typically, the natural product is plant or plant-derived material. For example, the natural product includes plants or plant parts including leaves, stems, roots, flowers, fruits, seeds or parts thereof. Typically, the natural product (or complex natural product) as described herein includes a mixture of one or more cannabinoids with other hydrophobic compounds, including but not limited to long-chain saturated and unsaturated fatty acids, fatty alcohols, fatty aldehydes, hydrocarbons, wax esters, sterols, terpenes etc.

In certain embodiments, the natural product that is used as starting material in the method of the invention is non-extracted material. For example, the natural product may be freshly harvested plant material. In such embodiments, the method of the invention may further comprise a pre-treatment step in which the plant material is dried to remove excess moisture. In certain embodiments, the pre-treatment step (e.g. heating) may also lead to decarboxylation of the one or more cannabinoids (e.g. to convert cannabinoid acids to the corresponding free cannabinoids).

In certain embodiments, the natural product that is used as starting material in the method of the invention is dried plant material.

In certain embodiments, the natural product that is used as starting material in the method of the invention is extracted material. For example, the natural product may be a crude plant extract (e.g. a concentrated fraction of plant material comprising one or more cannabinoids).

In certain embodiments, the natural product may have previously been extracted in a solvent such as alcohol, acetone, hexane or supercritical CO₂. Such extracts may have been dried (e.g. by evaporation of the solvent) to yield a soft extract as the starting material. The concentration of the starting extract is typically known. Typically, the starting extract is adjusted to a desired level when contacted (e.g. dissolved) with a known volume of the primary solvent as described herein.

In preferred embodiments, the plant is Cannabis (or the plant-material is derived from Cannabis). As used herein, the term “Cannabis” encompasses wild type Cannabis sativa and variants thereof including Cannabis indica, ruderalis or kafiristanica.

The term “Cannabis” includes chemovars known to contain desirable level(s) of one or more cannabinoids as described herein. The term “Cannabis” includes herbal Cannabis and/or dried Cannabis biomass. In preferred embodiments, the Cannabis is a variety or chemovar that has a high content of CBD and/or CBDA as compared to other cannabinoids such as THC. Methods of growing medicinal Cannabis and testing for content of one or more Cannabinoids are described in the art. Typically, the Cannabis is hemp or industrial hemp (e.g. a variety of Cannabis having 0.3% or less THC).

In preferred embodiments, the natural product is dried hemp (e.g. hemp dust) or a hemp extract (e.g. liquid extract obtained from “hemp tops” as described herein).

Primary Solvents

In the method of the invention, the natural product is contacted with a primary solvent to obtain a feed solution (e.g., liquid phase). Typically, the natural product is mixed and/or dissolved in a known volume of primary solvent.

In certain embodiments wherein the natural product is non-extracted material, contacting the non-extracted material (e.g. dried hemp) with the primary solvent provides a crude extract which dissolves within the solution. As such, the invention provides a “one-pot” system of extraction and purification as further described herein. Typically, any insoluble material resulting from contact with the primary solvent may be separately removed (e.g. by filtration).

In certain embodiments, the claimed method involves a step of collecting the insoluble material. For example, such “waste” material may also contain high-value compounds such as terpenes.

In certain embodiments wherein the natural product is an extracted material, contacting the extracted material (e.g. hemp extract) with the primary solvent also provides a dissolved solution.

In certain embodiments, a known amount of natural product (e.g. hemp extract) is contacted with a known amount of primary solvent to obtain a feed solution. A suitable ratio may depend on the type of starting material, type of cannabinoids being isolated, flow rates, temperatures and/or scale of the system.

The concentration of extract within the feed solution may depend, for example, on the natural product used as starting material, the type of cannabinoid(s) to be isolated, type of mesoporous material, flow rate to be used and/or scale of the system.

In certain embodiments, the extract within the feed solution is at a concentration of about 1.0 mg/mL (e.g. 53 g adsorbent per g of extract) to about 10 mg/mL (e.g. 5.3 g adsorbent per g of extract). In certain embodiments, the feed solution is at a concentration of about 1.0 mg/mL to about 7.5 mg/mL. For example, the extract within the feed solution may be at a concentration of about 1.25 mg/mL (e.g. 43 g absorbent per g of extract).

At commercial scale, the extract within the feed solution may be at a concentration higher than 1.25 mg/mL. For example, the extract within the feed solution may be at a concentration of about 5 mg/mL, about 10 mg/mL, about 15 mg/mL, about 20 mg/mL, about 50 mg/mL, about 100 mg/mL or more. The concentration may also be adjusted, for example, depending on the desired flow rate and/or type (or amount) of mesoporous material being used.

Any suitable primary solvent may be used in the methods of the invention. The type of primary solvent may depend on the natural product used as the starting material, the type of cannabinoid(s) to be isolated, type of mesoporous material and/or scale of the system. Typically, the primary solvent is a liquid or has liquid properties. Typically, the feed solution obtained by contacting the natural product (or extract thereof) with the primary solvent is liquid.

In preferred embodiments, the primary solvent is non-polar. Typically, the one or more cannabinoids have a lower affinity to the non-polar solvent as compared to the surface of the mesoporous material used in the methods of the invention.

Any non-polar solvent capable of solubilising the one or more cannabinoids (including their acids) may be used. Suitable non-polar solvents include liquid non-polar solvents comprising lower C5 to C12 (preferably C5 to C8) straight chain or branched chain alkanes. Typically, the non-polar solvent is hexane.

In certain embodiments, the primary solvent is mixed with water. The primary solvent may have low lipophilicity. The primary solvent may be treated with one or more chemicals prior to contact with the natural product. For example, the pH may be adjusted.

In preferred embodiments, the non-polar solvent is supercritical CO₂. Advantageously, contacting the natural product with carbon dioxide avoids the use of hazardous chemicals or solvents such as hexane.

Extraction of plant material using supercritical CO₂ is described in the art. This technique involves the use of carbon dioxide when held at or above its critical temperature (e.g. about 31.0° C.) and critical pressure (e.g. about 73.8 bar). As further described herein, the natural product may be contacted with carbon dioxide at a temperature of about 50° C. and a pressure of about 350 bar. Typically, a flow rate of about 30 g min⁻¹ of carbon dioxide is applied to the natural product for up to approximately 2 hours.

As further described herein, the feed solution obtained by contacting the natural product with the primary solvent is liquid and is subsequently passed through one or more solid mesoporous materials.

Mesoporous Material

Any mesoporous material allowing adsorption of the one more cannabinoids when dissolved in the primary solvent may be used.

The mesoporous materials are solid (i.e. solid phase). For example, the mesoporous materials may be a powdered composition. In certain embodiments, the mesoporous material is housed within a column (i.e. cartridge or plug) as further described herein.

The mesoporous material may be of any suitable material, including, for example, silica, alumina, zirconia, zeolites, carbon and the like.

In certain embodiments, the feed solution is passed through a mixture of mesoporous and microporous materials.

As used herein, the terms “mesoporous” and “microporous” are used in accordance with IUPAC (International Union of Pure and Applied Chemistry) standards. Mesopores include pore size distributions typically between 2 to 50 nm (20 to 500 A) whereas materials with pore sizes typically smaller than 2 nm (20 A) are considered as microporous.

In certain embodiments, the mesoporous material has a high mesopore volume (Vₘₑₛₒ) e.g. about 0.2 to about 2.0 cm³ g⁻¹, typically about 0.5 to about 2.0 cm³ g⁻¹.

In certain embodiments, the mesoporous material has a high surface area e.g. at least about 150 m² g⁻¹, about 200 m² g⁻¹, about 250 m² g⁻¹ or more, typically up to about 800 m² g⁻¹. Porosity and surface areas can be measured, for example, using automated BET measuring devices. Techniques such as electron microscopy and spectroscopic probing can be used to study the surface structure, energy and chemistry of mesoporous material.

In certain embodiments, the mesoporous material has pores in which a ratio of the mesoporous volume (Vₘₑₛₒ) to a microporous volume (V_{micro}) is greater than about 5, about 10, about 15, about 20 or more.

In certain embodiments, the bed depth of mesoporous material within a column (e.g. cartridge) is adjusted to optimise the flow of the primary solvent through the column (e.g. to prevent backwashing). Advantageously, this may improve recovery of the one or more cannabinoids.

The mesoporous materials used in the method of the invention may be prepared in any suitable way. For example, processes for preparing mesoporous carbons are well described in the art and include hard-templation. This technique comprises polymerisation of resin monomers around a sacrificial silica porogen having a shape and size of the desired pores. The carbonisation of the resulting resin-silica hybrid, and dissolution of the silica template yields mesoporous carbon. By controlling the parameters of the template, mesoporous carbons with the required pore size, surface areas and/or volumes can be produced.

As used herein “carbonisation” refers to a thermal treatment process of pyrolysis, in which the chemical structure of the material is modified following the thermal treatment process, such as by modification of, or loss of, functional groups.

In certain embodiments, the surface of the mesoporous material predominantly contains aromatic functionality. In certain embodiments, the mesoporous material functionality contains non-hexagonal aromatic rings. In certain embodiments (e.g. following thermal treatment at around 800° C.), the mesoporous material is fullerene-like.

In certain embodiments, the mesoporous material is functionalised with oxygen-containing groups. For example, the mesoporous material may be functionalised with hydroxyl (e.g. alcohol) groups. Typically, the mesoporous material has high surface polarity as compared to any equivalent non-functionalised material.

In certain embodiments, the mesoporous material is functionalised with carbonyls, aldehydes and/or carboxylic acids.

In preferred embodiments, the mesoporous material is polysaccharide-derived mesoporous carbon (Starbons™). Such material is commercially available from Starbons Limited. Such material is a bio-derived alternative to conventional mesoporous carbons. This material is prepared from mesoporous polysaccharide aerogels, which form due to the natural tendency of gelling polysaccharides to create extended 3D networks in water, i.e. gels. When dried appropriately, these gels retain the porous structure of the 3D network, resulting in mesoporous polysaccharide aerogels with textural properties (surface area, pore volumes, pore size) dependent on the type of polysaccharide used.

In certain embodiments, the mesoporous material is derived from polysaccharides having acid-functionality. For example, the mesoporous material may be derived from alginic acid and/or pectin. Mesoporous material derived from such polysaccharides typically possesses a high mesopore volume, high surface area and desirable surface properties. For example, mesoporous materials derived from polysaccharides having acid-functionality (e.g. alginic acid or pectin) may have lower micropore content than other polysaccharides which do not have acid functionality (e.g. starch).

Methods for preparing polysaccharide-derived mesoporous carbon suitable for use in methods of the invention are described, for example, in W02005/011836 and U.S. Pat. No. 5,958,589, the disclosures of which are hereby incorporated by reference. In particular, polysaccharide-derived mesoporous carbon may be prepared by (i) thermally assisted hydration of a polysaccharide to yield a polysaccharide/water gel or colloidal suspension, (ii) allowing the polysaccharide to recrystallise and (iii) exchanging the water in the recrystallised polysaccharide with a water miscible non-solvent for the polysaccharide which has a lower surface tension than water.

In certain embodiments, the mesoporous material is functionalised prior to thermal treatment for carbonisation. For example, oxidation of the mesoporous material may be used to introduce carboxylic acid groups. The high surface area polysaccharide derived porous material can be converted into a carbonised mesoporous material by heating in any suitable conditions. The heating may be carried out at any temperature or condition at which suitable modification of the expanded polysaccharide occurs including in particular, partial carbonisation, substantially complete carbonisation or complete carbonisation.

The mesoporous material can be functionalised or derivatised by any suitable technique. For example, the polysaccharide-derived mesoporous carbon may include chemically bound functional moieties and/or functional moieties immobilised within the material. Typically, the polysaccharide-derived mesoporous carbon comprise oxygen-containing groups. In preferred embodiments, the polysaccharide-derived mesoporous carbon comprises hydroxyl and/or aromatic groups. Such functional groups may act to improve adsorption of the one or more cannabinoids to the mesoporous material.

In certain embodiments, the mesopore material comprises Starbons A300. In such embodiments, the mesopore material typically comprises:
(i) pore sizes having an average diameter of about 18 nm;
(ii) mesopore volumes between about 0.2 cm3 g⁻¹ to about 0.9 cm3 g⁻¹; and/or
(iii) a surface area between about 50 m² g⁻¹ to about 216 m² g⁻¹.

In certain embodiments, the mesopore material comprises Starbons P300. In such embodiments, the mesopore material typically comprises:
(i) pore sizes having an average diameter of about 24 nm;
(ii) mesopore volumes between about 0.4 cm3 g⁻¹ to about 1.2 cm3 g⁻¹; and/or
(iii) a surface area between about 50 m² g⁻¹ to about 160 m² g⁻¹.

In certain embodiments, the mesopore material comprises Starbons P800. In such embodiments, the mesopore material typically comprises:
(i) pore sizes having an average diameter of about 14 nm;
(ii) mesopore volumes between about 0.2 cm3 g⁻¹ to about 0.4 cm3 g⁻¹; and/or
(iii) a surface area between about 400 m² g⁻¹ to about 800 m² g⁻¹.

In the method of the invention, the feed solution is typically passed through the mesoporous material under conditions allowing adsorption of the one or more cannabinoids to the mesoporous material. Any suitable conditions may be used. Typically, suitable conditions may depend on the natural product used as starting material, the type of cannabinoid(s) being isolated, the type of mesoporous material and/or the scale of the system.

In certain embodiments, the feed solution is passed through the mesoporous material at a pre-selected volume, flow rate, pressure and/or temperature. These operating parameters may be selected based on the physio-chemical composition of the feed solution and/or type of cannabinoids being recovered. As further described herein, these operating parameters may also depend on the type and/or amount of mesoporous material being used, including, for example, the bed depth of mesoporous material within a column.

In certain embodiments, the feed solution is passed through the mesoporous material at a flow rate that allows contact of the solution with the mesoporous material for between about 1 minute to about 5 minutes. Typically, the flow rate is adjusted based on the concentration of extracted plant material within the feed solution and/or the amount of mesoporous material being used.

For example, where larger bed depths are used at a commercial scale, the flow rate may be adjusted to be faster and/or the concentration of the feed solution may be increased. Adjusting such parameters may allow a suitable total quantity of extracted plant material within the feed solution to be contacted with the mesoporous material for the desired amount of time (e.g. between about 1 minute to about 5 minutes).

By way of example only, a total of approximately 18 kg extracted plant material at a concentration of about 1 mg/ml in the feed solution may be passed through the mesoporous material at a flow rate of between about 30 l^{s-1} to about 1 l^{s-1}. Typically, however, a slower flow rate may be used but at a higher concentration of feed solution (e.g. about 7.5 mg/ml or more).

In certain embodiments, the feed solution is passed through the mesoporous material at a flow rate of about 30 ml^{-s-1}, about 20 ml^{-s-1}, about 10 ml^{-s-1}, about 5 ml^{-s-1}, about 1 ml^{-s-1} or less. In certain embodiments, the feed solution is passed (e.g. circulated) through the mesoporous material under pressure or by applying vacuum as the driving force.

In certain embodiments, the mesoporous material is washed one or more times with a wash solution (e.g. the primary solvent alone) prior to passing the feed solution through the mesoporous material.

In certain embodiments, the amount and/or type of cannabinoid(s) are determined in the feed solution after passing through the mesoporous material. Techniques for measuring the amount and/or type of cannabinoids are known in the art. If the feed solution still contains high amounts of one or more cannabinoids after passing through the mesoporous material, it may be re-passed through the mesoporous material until more cannabinoid(s) are adsorbed onto the mesoporous material and out of the feed solution.

In certain embodiments, the solution is passed repeatedly (e.g. 2, 3, 4, 5 times or more) through the one or more mesoporous materials.

In certain embodiments, the feed solution is passed through one or more columns comprising the mesoporous material. For example, the columns may comprise an outer housing that contains the mesoporous material within. In such embodiments, the feed solution may be passed into an inlet of the housing and through the mesoporous material under conditions that allow adsorption of the one or more cannabinoids to the mesoporous material. The solution may then be passed into an outlet of the housing allowing recovery of the solution. Typically, the columns are plugs and/or cartridges. Typically, the mesoporous material is used as a stationary phase or filter material within the column. Typically, the columns contain about 1 g, about 5 g, about 10 g, about 15 g, about 20 g or more of mesoporous material (e.g. powdered composition).

In certain embodiments, the bed depth of mesoporous material is adjusted to optimise flow through the column.

In certain embodiments, at least one column volume of the feed solution is passed through the mesoporous material.

In certain embodiments, more than one column volume (e.g. 2, 3, 4, 5 times or more) of the feed solution is passed through the mesoporous material.

As used herein, the “column volume” refers to the volume inside of the column (e.g. cartridge) not occupied by the mesoporous material. This volume includes both the interstitial volume (e.g. volume outside of the mesoporous material) and the mesoporous material's internal porosity (total pore volume). As such, the total of volume of liquid passed through the column will typically depend on the size of column used and the amount of mesoporous material within the column.

In certain embodiments, the concentration and volumes of material are scaled to mimic 0.3 ml of the feed solution at 1.25 mg mL⁻¹ being passed through an 18 mg cartridge. By way of example, approximately 167 ml of solution at 1.25 mg mL⁻¹ may be passed through a 10 g cartridge. At commercial scale, however, less solvent is typically used at a higher concentration.

In certain embodiments, the method is operated continuously and is particularly suitable for use in large scale commercial production of one or more cannabinoids.

Washing Steps

In the method of the invention, one or more wash solutions may be passed through the mesoporous material. The mesoporous material may be washed before and/or after the feed solution is passed through the mesoporous material.

In certain embodiments, one or more wash solutions are passed through one or more columns comprising the mesoporous material as described herein.

In certain embodiments, one or more wash solutions are passed through the mesoporous material before the feed solution has been passed through the mesoporous material, e.g. before any cannabinoid(s) are bound to the material.

In certain embodiments, one or more wash solutions are passed through the mesoporous material after the feed solution has been passed through the mesoporous material, e.g. whilst cannabinoid(s) are bound to the material.

Advantageously, washing the mesoporous material ensures only compounds which are strongly adsorbed to the mesoporous material remain in the system.

In certain embodiments, the mesoporous material is repeatedly washed (e.g. 2, 3, 4, 5 times or more) with one or more wash solutions prior to passing the second solvent through the mesoporous material. Where there are multiple washes, the same type and/or concentration of wash solution may be used in each wash step. Alternatively, different types and/or concentrations of wash solution may be used for each wash step.

Any suitable wash solution(s) may be used. Typically, the wash solution is a solvent (e.g. non-polar solvent). Typically, the cannabinoid(s) has a lower affinity to the “wash” solvent as compared to the surface of the mesoporous material that is used. Typically, the wash solution is liquid. Typically, the wash solution is the same as the primary solvent.

In certain embodiments, the wash solution is passed through the mesoporous material(s) under conditions that act to maintain or increase adsorption of the cannabinoid(s) to the mesoporous material. Again, the conditions may vary depending on the natural product used as starting material, the type of cannabinoid(s) being isolated, the type of mesoporous material and/or the scale of the system.

In certain embodiments, the wash solution is passed through the mesoporous material at a pre-selected volume, flow rate, pressure and/or temperature. These operating parameters may be selected, for example, based on the physio-chemical composition of the wash solution and/or type of cannabinoids to be recovered.

The operating parameters of the wash steps typically correspond to those used for passing the feed solution through the mesoporous material as described herein. For example, the wash solution may be passed through the mesoporous material at a flow rate that allows contact of the second solvent with the mesoporous material for between about 1 minute to about 5 minutes as further described above.

In certain embodiments, at least one column volume of the wash solution is passed through the mesoporous material.

In certain embodiments, more than one column volume (e.g. 2, 3, 4, 5 times or more) of the wash solution is passed through the mesoporous material.

In certain embodiments, the wash solution is passed through the mesoporous material(s) at room temperature or lower, e.g. at about 24° C., about 20° C., about 10° C. or less.

In certain embodiments, the wash solution is passed (e.g. circulated) through the mesoporous material under pressure or by applying vacuum as the driving force.

Desorption Steps

In the method of the invention, a second solvent is passed through the mesoporous material containing one or more adsorbed cannabinoids. Passing the second solvent through the mesoporous material allows desorption of the cannabinoid(s) from the mesoporous material allowing their subsequent recovery.

In certain embodiments, the second solvent is passed through one or more columns comprising the mesoporous material as described herein.

In certain embodiments, the second solvent is passed repeatedly (e.g. 2, 3, 4, 5 times or more) through the one or more mesoporous materials.

Any suitable second solvent may be used in the methods of the invention. The second solvent is different than the first solvent. The second solvent that is used may depend on the natural product as starting material and/or the type of cannabinoids to be isolated. Typically, the second solvent is liquid.

In preferred embodiments, the secondary solvent is polar. Typically, the one or more cannabinoids have greater affinity to the polar solvent as compared to the surface of the mesoporous material used in the methods of the invention.

Any second solvent capable of desorbing the one or more cannabinoids (including their acids) may be used. Typically, the second solvent is an alcohol e.g. methanol, ethanol or 2-propanol. Preferably, the second solvent is ethanol. Advantageously, the use of ethanol is environmentally friendly as compared to certain other types of alcohol.

In certain embodiments, the second solvent is mixed with water. The second solvent may have reduced lipophilicity. The second solvent may be subject to one or more chemical treatments. For example, the pH of the second solvent may be adjusted.

Any suitable conditions that allow desorption of the one or more cannabinoids from the mesoporous material may be used. Typically, the conditions that are chosen may depend on the natural product used as starting material, the type of cannabinoid(s) being isolated, the type of mesoporous material and/or the scale of the system.

In certain embodiments, the second solvent is passed through the mesoporous material at a pre-selected flow rate, pressure and/or temperature. These operating parameters may again be selected based on the physio-chemical composition of the solvent and/or type of cannabinoids being recovered from the mesoporous material.

In certain embodiments, the second solvent is passed through the mesoporous material at a controlled temperature, flow rate and/or pressure.

In certain embodiments, the second solvent is passed through the mesoporous material at a similar flow rate as the first solvent. For example, the second solvent may be passed through the mesoporous material at a flow rate that allows contact of the second solvent with the mesoporous material for between about 1 minute to about 5 minutes as further described above.

In certain embodiments, at least one column volume of the second solvent is passed through the mesoporous material.

In certain embodiments, more than one column volume (e.g. 2, 3, 4, 5 times or more) of the second solvent is passed through the mesoporous material.

In certain embodiments, the second solvent is passed (e.g. circulated) through the mesoporous material under pressure or by applying vacuum as the driving force.

In the method of the invention, contacting the mesoporous membranes with the second solvent allows the cannabinoid(s) to dissolve into an elution solution which may then be separately recovered.

In certain embodiments, the content of one or more cannabinoids is determined in the elution solution using any suitable technique. For example, if the level of cannabinoid(s) is low, the second solvent may be re-passed through the mesoporous material until a desirable level of cannabinoid(s) is reached.

In the method of the invention, an elution solution is obtained by passing the second solvent through the mesoporous material.

In certain embodiments, the second solvent is removed from the elution solution using any suitable technique (e.g. by drying or evaporation).

Typically, the elution solution comprises a cannabinoid-rich extract as further described herein.

Re-Conditioning of Mesoporous Material

In certain embodiments, the method of the invention comprises re-conditioning the mesoporous material for re-use. Typically, the mesoporous material is housed within one or more columns as described herein.

In certain embodiments, the method of the invention further comprises removing any second solvent that may be retained on the mesoporous material after the second solvent has been passed through the mesoporous material. Any suitable technique may be used to remove the second solvent from the mesoporous material, including, for example, air or vacuum drying.

In certain embodiments, one or more of the wash solutions are passed through the mesoporous material after the second solvent has been passed through the mesoporous material. This allows repeated use of the mesoporous material for isolating one or more cannabinoids from natural products.

In certain embodiments, the invention provides use of any mesoporous material as described herein in isolating cannabinoid(s) from natural products as described herein. Typically, the cannabinoid(s) are CBD and/or CBDA as described herein. Typically, the mesoporous material is functionalised with aromatic and/or hydroxyl groups as described herein. This allows effective recovery of the cannabinoid(s) using non-polar adsorption solvents and polar desorption solvents as described herein.

Cannabinoid-Rich Extract

In certain embodiments, the invention provides a cannabinoid-rich extract obtained by the method described herein.

In certain embodiments, the elution solution obtained by passing the second solvent through the mesoporous material is further treated to produce the cannabinoid-rich extract. For example, the second solvent may be removed by evaporation and/or drying. In such embodiments, the cannabinoid-rich extract may be in crystalline form (e.g. a white crystalline form, solid at room temperature).

In certain embodiments, one or more desired cannabinoids (or acids thereof) of the cannabinoid-rich extract have a chromatographic purity of greater than 80%, greater than 90%, greater than 95%, greater than 99% or greater than 99.5%, as determined, for example, by area normalisation of a HPLC profile.

In certain embodiments, the cannabinoid-rich extract comprises purified CBD and/or CBDA. In such embodiments, the cannabinoid-rich extract may also comprise detectable levels of CBG, CBC and/or THC as measured by gas-chromatography-electron impact-mass spectrometry (GC-EI-MS) analysis. The cannabinoid-rich extract is therefore distinct from extracts obtained by supercritical carbon dioxide without solid phase extraction using mesoporous materials, where these additional cannabinoids are not detectable.

In certain embodiments, the invention provides a cannabinoid-rich extract, wherein the extract has the GC-EI-MS profile substantially as shown in FIG. 25.

In certain embodiments, the cannabinoid-rich extract comprises purified CBD and/or CBDA and comprises less than about 5%, less than about 1%, less than about 0.2% or less than about 0.1% CBG.

In certain embodiments, the cannabinoid-rich extract comprises purified CBD and/or CBDA and comprises less than about 5%, less than about 1%, less than about 0.2% or less than about 0.1% CBC.

In certain embodiments, the cannabinoid-rich extract comprises purified CBD and/or CBDA and comprises less than about 5%, less than about 1%, less than about 0.2% or less than about 0.1% THC.

In certain embodiments, the cannabinoid-rich extract has a melting point of about 60° C. to 70° C., e.g. about 65° C.

Pharmaceutical Compositions

Advantageously, the cannabinoid(s) obtained by the method of the invention have a range of pharmaceutical properties and may be used to treat, for example, neurological and central nervous system (CNS) disorders, seizures, spasms, migraines, pain relief, anxiety or glaucoma. The cannabinoids may also have beneficial anti-nausea, anti-bacterial and/or anti-inflammatory properties.

In certain embodiments, the invention provides a pharmaceutical composition comprising the cannabinoid-rich extract as described herein.

In certain embodiments, the invention provides a method of making a pharmaceutical composition comprising (i) isolating one or more cannabinoids according to the method as described herein, and (ii) formulating the isolated cannabinoid(s) with one or more pharmaceutically acceptable diluents, carriers or excipients.

In certain embodiments, the one or more cannabinoids obtained by the method of the invention are at a sufficient purity to be processed directly into pharmaceutical dosage forms (e.g. tablets, capsules, sprays, liquid formulations and the like).

In certain embodiments, the one or more cannabinoids obtained by the method of the invention are further purified, e.g. CBD and/or CBDA may be further isolated from the cannabinoids by any suitable technique (e.g. chromatography steps). The cannabinoid content may be qualitatively and quantitatively determined using analytical techniques well known to those skilled in the art, such as thin-layer chromatography (TLC, high performance liquid chromatography (HPLC) or the like.

### EXAMPLES

In the following, the invention will be explained in more detail by means of non-limiting examples of specific embodiments. In the example experiments, standard reagents and buffers free from contamination are used.

### Example 1

Materials and Methods

Chemicals

Hexane, cyclohexanone and propanol were purchased from Sigma Aldrich. Methanol, ethanol and 2-propanol were purchased from Fisher Chemical. Deuterated chloroform was purchased from Sigma Aldrich.

Biomass

Hemp tops (HTs) were provided by Adact Chemical ltd. The US variety of hemp was obtained for this work due to the high levels of CBDA (ca. 20% w/w). Hemp dust (HD) was obtained from a hemp processing facility in North Yorkshire, UK.

Starbons

The solid-phase extraction cartridges for the autosampler were obtained from ITSP Solutions Inc. These were packed with 18 mg of Starbon prepared at the Green Chemistry Centre of Excellence (University of York, UK). Starbons used for packing were made from starch, alginic acid or pectin. Alginic acid-based Starbons (A-series) were synthesised through Starbons Ltd at the biorenewables development centre (BDC) in Dunnington, York and are derived from kelp. Starch based Starbons (S-series) were produced in house and derived from potatoes. Pectin based Starbons (P-series) were produced in house and derived from orange peel waste. Different Starbons were used at varied preparation temperatures: A-series 0, 300, 450 and 800° C.; S-series 0, 300, 450 and 800° C.; and P-series 300 and 800° C. across this work.

Auto Sampled Adsorption and Desorption of Crude Hemp Extracts

Hemp extracts (20 mg) were dissolved in hexane (20 mL) and placed on a Gerstel multipurpose sampler (MPS) by Anatune. Prior to adsorption/desorption experiments, 300 μL of 0.1 mg mL⁻¹ cyclohexanone in 2-propanol was added to the gas chromatography (GC) vials as an external standard for quantification.

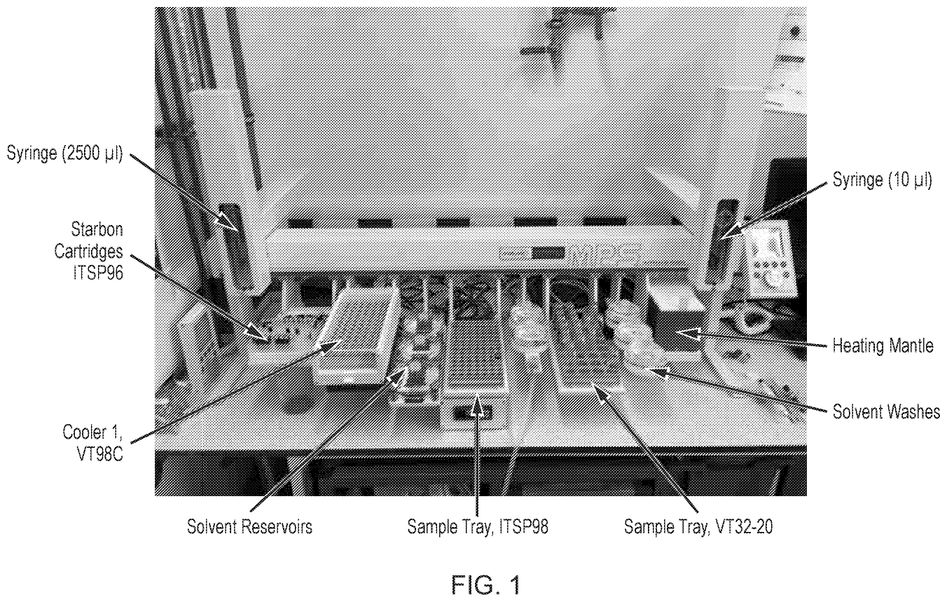

External standard addition was carried out on the chilled Cooler1 on the autosampler (FIG. 1). The external standard was added into the designated vials and then the following washing steps were done in methanol, ethyl acetate then hexane. This is summarised in Table 1 below.

TABLE 1
Method of addition of the external standard.

| | | | | | | |
|---|---|---|---|---|---|---|
| | | | Add | Eject | | |
| | | | speed | speed | | |
| Sequence | Action | Method | (μLs⁻¹) | (μLs⁻¹) | Source | Destination |

Start

| | | | | | | |
|---|---|---|---|---|---|---|
| 1 | Add | Add 300 μL | 250 | 250 | Tray1, | Cooler1, |
| | | Standard | | | VT32-20 | VT98C |
| 2 | Add | Wash Waste | 250 | 250 | Wash 5 | Waste |
| 3 | Add | Wash Waste | 250 | 250 | Wash 3 | Waste |
| 4 | Add | Wash Waste | 250 | 250 | Wash 4 | Waste |

End

Post-external standard addition, the GC vials were moved manually from the cooler rack to the sample tray ITSP98 (FIG. 1). Each repetition on the autosampler consisted of three key steps: adsorption, washing and desorption. Any Starbon cartridge used for a particular adsorption/desorption set of experiments was flushed with hexane immediately prior to the run to condition the adsorbent. Hemp extract (300 μL) was removed by the 2500 μL syringe, passed through the ITSP cartridge, and the adsorption phase collected. The cartridge was then flushed with 500 μL hexane to ensure only compounds that have been strongly physisorbed by the Starbon remain in the system. Sequential washing of the needle was carried out with 500 μL of methanol, ethyl acetate then hexane to ensure no contamination of the desorb solvent occurs. Subsequently, 300 μL of methanol was used to prime the syringe and an additional 300 μL of methanol was then passed through the cartridge and collected to give the desorption phase. The cartridge was then flushed with 500 μL of methanol to ensure total desorption of physisorbed constituents from the Starbon. The syringe was washed with 500 μL hexane to clean it, and the ITSP cartridge flushed with 500 μL hexane to condition for the next run. For more details see Table 2 below where sequence 2-5 concerns the adsorption of hemp extract onto the Starbon. Sequence 12-15 denotes desorption of hemp material from the Starbon cartridge (Table 2).

TABLE 2
Optimised method for adsorption and desorption run for extractions
of scCO₂ hemp. Using hexane/methanol as solvents.

| | | | | | | |
|---|---|---|---|---|---|---|
| | | | Add | Eject | | |
| | | | speed | speed | | |
| Sequence | Action | Method | (μLs⁻¹) | (μLs⁻¹) | Source | Destination |

Start

| | | | | | | |
|---|---|---|---|---|---|---|
| 1 | Add | Rinse 500 μL | 10 | 250 | SolvRes2 | ITSPprepITSP96 |
| 2 | Add | Add 300 μL | 10 | 250 | Tray1, VT32-20 | Syringe |
| 3 | Move | | | | ITSPprepITSP96 | Tray |
| | | | | | | 2ISPVT98elut |
| 4 | Add | ITSP elute | 10 | 250 | Syringe | Tray |
| | | 300 μL | | | | 2ISPVT98elut |
| 5 | Move | | | | Tray | ITSPprepITSP96 |
| | | | | | 2ISPVT98elut | |
| 6 | Add | Wash waste | 250 | 250 | Wash 1 | Waste |
| 7 | Add | Wash waste | 250 | 250 | Wash 3 | Waste |
| 8 | Add | Wash waste | 250 | 250 | Wash 4 | Waste |
| 9 | Add | Wash waste | 250 | 250 | SolvRes2 | Waste |
| 10 | Add | Rinse 500 μL | 10 | 250 | SolvRes2 | ITSPprepITSP96 |
| 11 | Add | Wash waste | 250 | 250 | SolvRes3 | Waste |
| 12 | Add | Add 300 μL | 250 | 250 | SolvRes3 | Syringe |
| 13 | Move | solvent | | | ITSPprepITSP96 | Tray |
| 14 | Add | ITSP elute | 10 | 250 | Syringe | Tray |
| | | 300 μl | | | | 2ISPVT98elut |
| 15 | Move | | | | Tray | ITSPprepITSP96 |
| | | | | | 2ISPVT98elut | |
| 16 | Add | Wash Waste | 250 | 250 | SolvRes3 | Waste |
| 17 | Add | Wash Waste | 250 | 250 | SolvRes2 | Waste |
| 18 | Add | Rinse 500 μL | 10 | 250 | SolvRes2 | ITSPprepITSP96 |

End

The above method was repeated several times using different Starbon cartridges: S300, S450, S800 and A300, A450, A800, P300, P800 & AC.

Scale-Up of A300 and P300

Larger scale adsorption/desorption isolations were run using A300 and P300.

A300 Scale-Up

207 mg of HT extract was dissolved in 166 mL of hexane to give a 1.25 mg mL⁻¹ solution, which was pulled through a plug of A300 Starbon (10 g) using a vacuum line and the adsorption phase collected. The cartridge was then washed with 166 mL of hexane to ensure all compounds not physisorbed to the support had been removed. The ethanol desorption solvent (166 mL) was subsequently passed through the Starbon and collected. This was then washed sequentially with ethanol and hexane (166 mL) to prepare for additional runs. The adsorption/desorption run was done twice. The desorption solvent was removed in vacuo to afford products to undergo further analysis. Aliquots of each phase were collected and external standard was added to quantify the results. All masses and volumes were scaled to mimic 0.3 ml of solution at 1.25 mg mL⁻¹ being passed through an 18 mg Starbon cartridge.

P300 Scale-Up

62.5 mg of HT extract was dissolved in 50 mL of hexane for a 1.25 mg mL⁻¹ solution, which was pulled through a plug of P300 Starbon (3 g) using a vacuum line and the adsorption phase collected. The cartridge was then washed with 50 mL of hexane to ensure all compounds not physisorbed to the support had been removed. The ethanol desorption solvent (50 mL) was subsequently passed through the Starbon and collected. This was then washed sequentially with ethanol and hexane (50 mL) to prepare for additional runs. The adsorption/desorption run was done twice. The desorption solvent was removed in vacuo to afford products to undergo further analysis. Aliquots of each phase were collected, and external standard was added to quantify the results. All masses and volumes were scaled to mimic 0.3 mL of solution at 1.25 mg mL⁻¹ being passed through an 18 mg Starbon cartridge.

One-Pot scCO₂ Extraction/Starbon Isolation

Supercritical CO₂ has already been established as a viable methodology for extraction of hydrophobic constituents containing cannabinoids from various hemp sources. Initial proof of concept has already been demonstrated using hemp top extract loaded onto celite. Herein the direct isolation of cannabinoids from hemp dust in one pot is described.

Lab-Scale Supercritical Fluid Extraction of Hemp Biomass

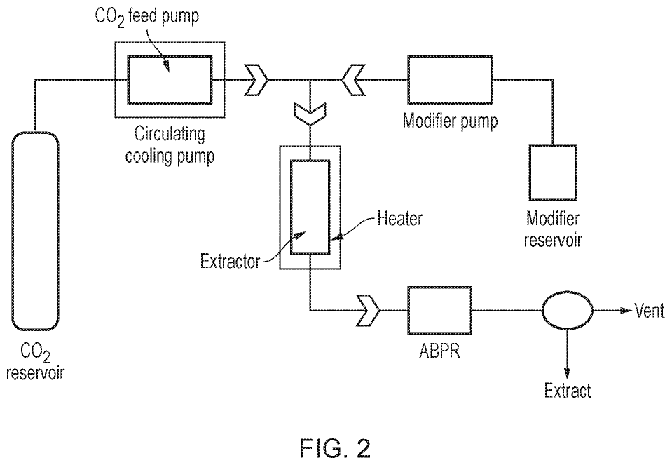

All scCO₂ extractions were carried out using an SFE-500 extractor provided by Thar technologies. FIG. 2 represents simplified scheme of extractor. 60 g of milled hemp biomass was placed into the 500 cm3 extraction vessel and connected to the extraction system. Liquid CO₂ was then pumped to the required pressure (maintained by an automated back pressure regulator—ABPR) and passed through an inline pre-heater maintained at the desired extraction temperature. The system was run for 2 hours at a flow rate of 30 g min⁻¹ CO₂ with continual collection of extract.

One-Pot System

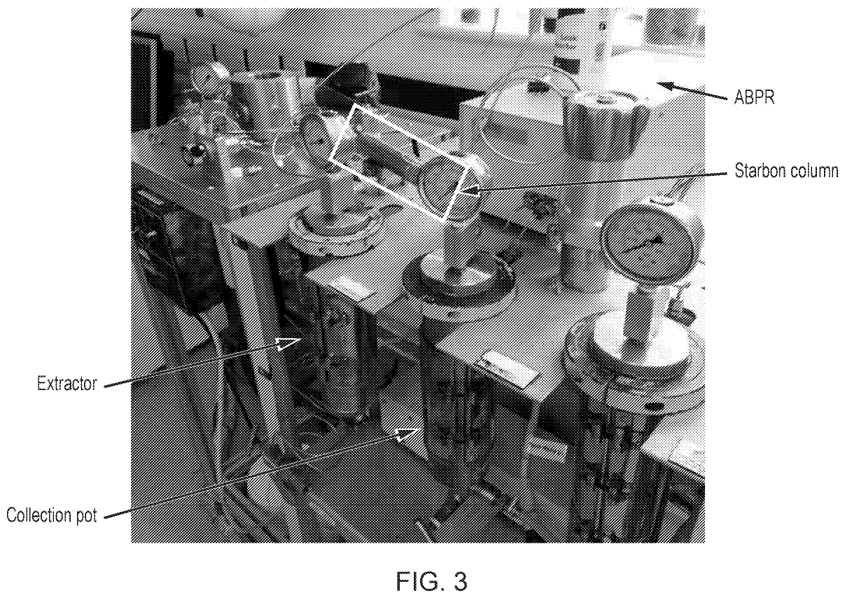

In the one-pot system, a stainless steel 10 cm3 column was filled with 8 g of Starbon A300 and connected between the extractor and the ABPR (FIG. 3). The system was set to the desired temperature and pressure and run for 2 hours at 30 g min⁻¹ and the extract collected and analysed. Post extraction, ethanol was passed through the column for 10 minutes using the co-solvent modifier pump at a 10 ml min⁻¹ flowrate to give the desorbed fraction. The column was reconditioned with scCO₂ at 120 bar, 50° C. for 30 minutes at 30 g min⁻¹.

Analysis of Hemp Extracts and Adsorption/Desorption Products

Gas Chromatography Flame Ionisation Detection (GC-FID)

Samples were quantified by using an Agilent Technologies 7890B GC system and a Hewlett Packard HP 6890 Series GC system. Both GCs were run using flame ionisation detection methods and on identical methods. A Rxi-5HT capillary column (30 m×250 μm×0.25 μm nominal) was fitted at constant pressure of 20.16 psi. Helium was the carrier gas used. Both the injector temperature and FID detector temperature were set at 320° C. 1 μl samples were injected by automated injection, with a split ratio of 5:1. The oven temperature profile was as follows: Initial temperature of 50° C., increased to 300° C. at a rate of 30° C. min⁻¹, held at this temperature for 5 mins. Quantification was carried out by means of an external standard (cyclohexanone).

Gas Chromatography Mass Spectrometry (GC-MS)

Gas chromatography mass spectrometry (GC-MS) was run on a Perkin Elmer Glarus 500 GC coupled with a Glarus 560 S mass spectrometer. This was run using a Rxi-5HT 30 m×250 μm×0.25 μm at a pressure of 21.4 psi. The carrier gas was helium. The injector temperature is set to 300° C. and the flow rate of 1.0 mL/min. The method was run at 50° C. starting temperature. The Glarus 500 quadrupole mass spectra were operated in the electron ionisation mode (EI+) at 70 eV, a source temperature of 300° C., quadrupole in the scan range of 30-1200 amu per second.

Starbons

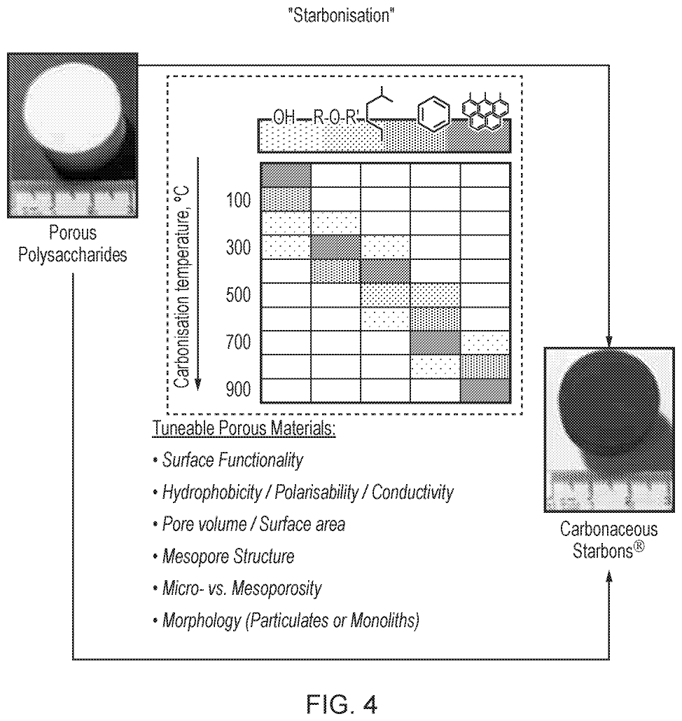

Starbon® is a registered trademark of Starbons Ltd and refers to mesoporous carbonaceous materials. These materials are produced from any polysaccharide, principally starch (from potatoes), alginic acid (from kelp) or pectin (from oranges) in a 3 stage process which consists of 1) gelation 2) drying 3) pyrolysis. The gelling stage allows the production of porous networks from which the mainly mesoporous systems of the final materials are derived. The gel is then dried in a way that does not collapse the porous system, before pyrolysing the material at the desired temperature. Depending on the starting material and thermal treatment, the surface chemistry and porosity of the Starbons can be tuned towards different applications (FIG. 4).

Results

Preliminary investigations involved assessing whether the Starbons were able to interact with the cannabinoids found in the hemp extracts, in a typical solid-phase extraction set-up. Upon selecting an adsorption solvent, the compound of interest must be soluble in the solvent; however, it must show a greater affinity for the adsorbent rather than the solvent. In contrast, in the case of a desorption solvent, the compound of interest must show a greater affinity for the solvent than the adsorbent.

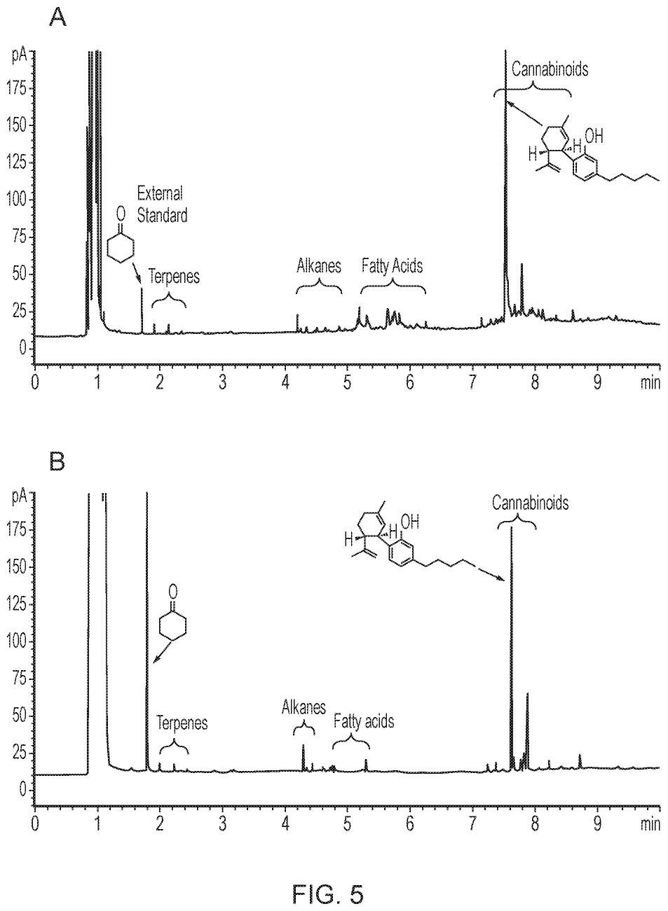

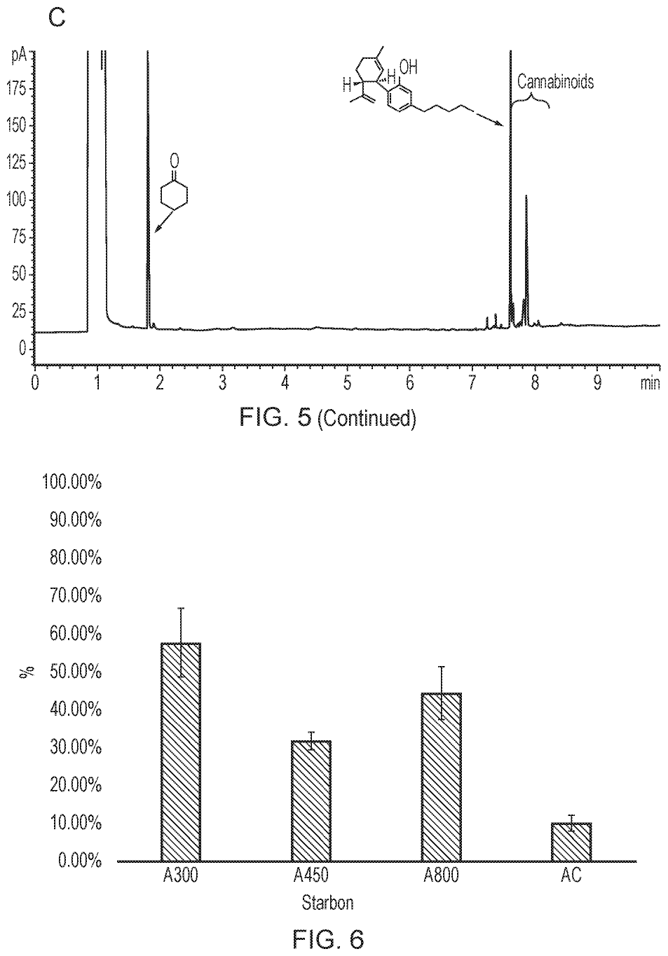

The adsorption solvent selected was hexane and the desorption solvent chosen was methanol. This is because a non-polar solvent is used to dissolve the HT extract so, when it passes through the cartridge, the CBD will have a greater affinity to the solid-phase surface chemistry of the Starbon cartridge than the liquid phase. The use of the desorption solvent must be sufficiently polar enough for the CBD molecule to have a greater affinity to the liquid phase and desorb off the Starbon surface. An Example of a GC chromatograph depicting the isolation of cannabinoids from Crude hemp extract can be seen in FIG. 5. Starbon S-Series (S0, S300, S450 and S800) and Starbon A-series (A0, A300, A450 and A800) were tested. However, all the S-series exhibited backpressure issues when trying to pass solvent through the packed-cartridges. The rate of adsorption and desorption of A300, A450 and A800 was analysed using GC-FID and compared to commercially available AC.

Results conducted on scCO₂ extracts from the HT extract indicated that all Starbons showed some degree of cannabinoid uptake. However, the A-series Starbon gave better adsorption and separation of cannabinoids than the S-series.

The S-series worked for one adsorption/desorption run; however, there were backpressure issues across the whole S-series. This suggested that the A-series was superior to the S-series as a result of increased mesoporosity and larger pore sizes in the A-series compared to the S-series, allowing for a greater adsorption capacity. Back pressure issues are also likely to be as a result of the difference in powder form of the two materials.

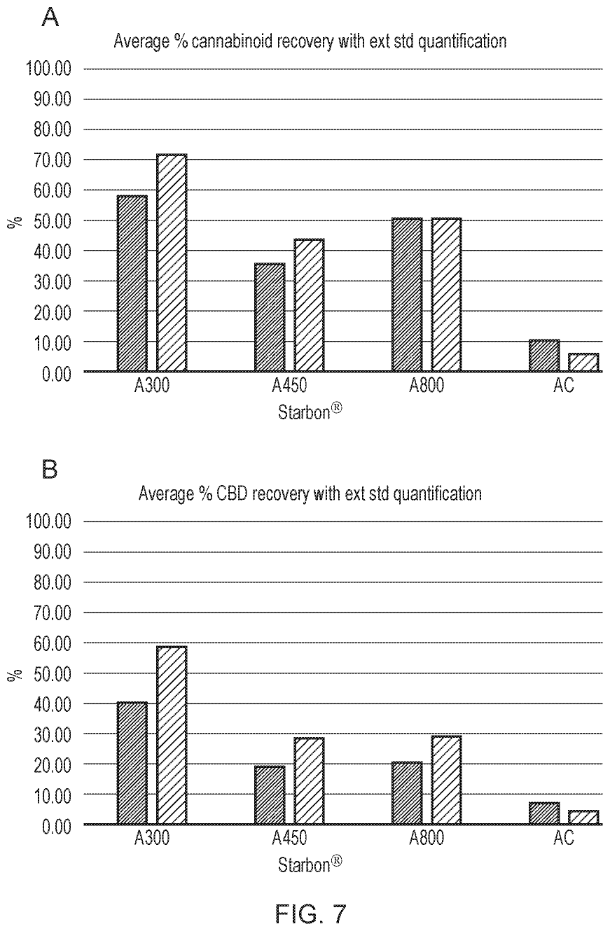

The AC exhibited poor recovery of CBD due to strong interactions of hemp moieties with micropores meaning desorption was very limited. FIG. 6 indicates the superiority of A300 in terms of CBD recovery, with a total extraction of 57.76% CBD. To confirm A300 was indeed best at CBD recovery, a number of repeat experiments were run, including a hexane pre-rinse on the cartridge and optimised washing steps. By implementing the cartridge preconditioning run and washing steps, the extractions yielded better recovery of CBD, increased purity and increased total cannabinoid recovery (FIG. 7). CBD recovery is increased for the Starbons and the AC showed no signs of improved extraction.

P series cartridges were also produced and the results were consistent with what was previously seen for A-series extraction. However, a significantly enhanced CBD recovery for P-series was observed compared to A-series Starbons. Both P300 and P800 showed excellent uptake of CBD with the almost all cannabinoids present in the extract being adsorbed by the Starbon. As with the A series, repeatability was confirmed by conducting 4 repeats on the same cartridge.

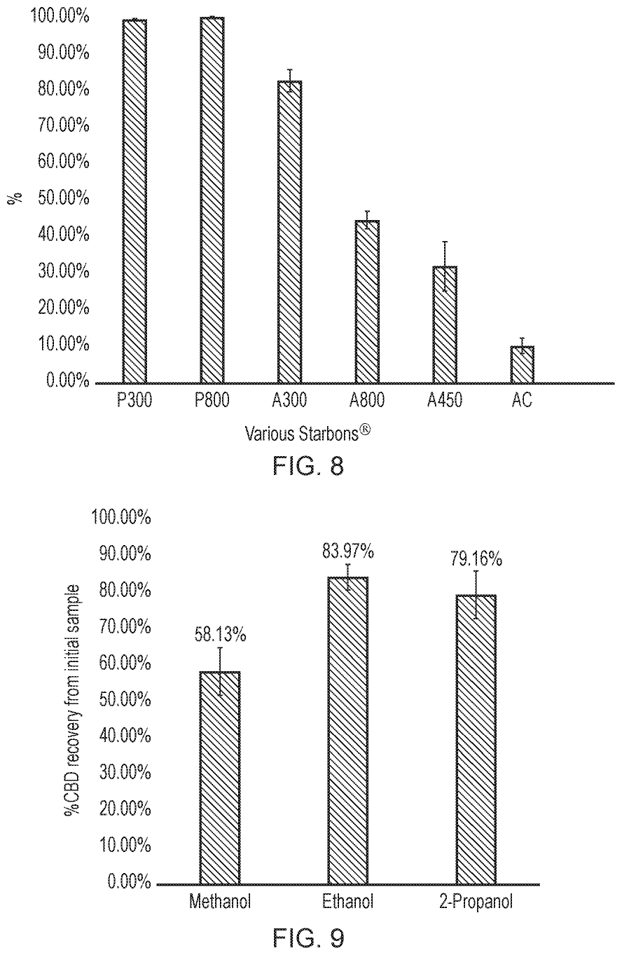

The P300 showed a 99±0.23% recovery of CBD and 93±0.11% cannabinoid recovery, P800 extracted 99±0.24% of CBD and 96% recovery of cannabinoids (FIG. 8). This better than results from the A-series Starbons, where the highest recovery obtained was 82.36±3.0%. The P-series exhibit greater CBD recovery than other mesoporous carbonaceous materials (FIG. 8). This could be due to the increased mesoporosity of the P-series allowing higher adsorption of CBD onto the Starbon.

Solvent Trials

Methanol is an efficient desorption solvent but has issues relating to toxicity and sustainability. Prat et al. denoted how methanol would rank poorly if designated metrics were used to measure environmental impact of the manufacturing of solvents, such as using CO₂ footprint (kg kg⁻¹). A range of different desorption solvents were investigated to determine whether CBD recovery is possible without the use of methanol. Non-polar hexane was kept as the adsorption solvent, with the polar solvents ethanol and 2-propanol trialed as desorption solvents. Ethanol was found to show the greatest CBD recovery at 83.97±3.5% and 2-propanol also showed an increased CBD recovery in comparison to the methanol (FIG. 9). The use of ethanol is desired as it is a common low-cost bio-solvent that can be obtained by the fermentation of renewable biomass. The implementation of ethanol in this work does bring risks as it is flammable and potentially explosive. However, it is completely biodegradable, a food-grade solvent and has a very high solvating power that can be used for purification of natural products.

In addition to increased CBD recovery, ethanol also gave improved total cannabinoid recovery. Although the desorption of 2-propanol could be seen to be almost as effective as ethanol, the latter has more preferential green properties.

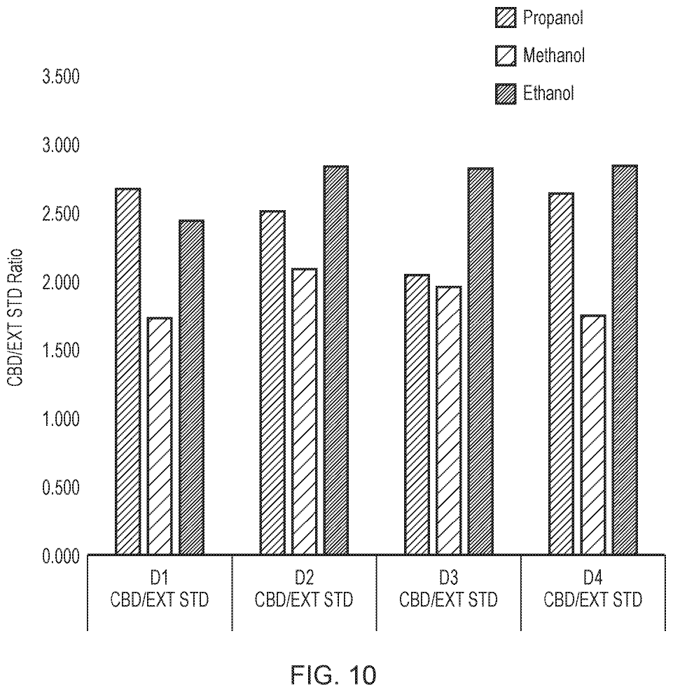

The “D” in FIG. 10 represents the desorption run and shows CBD that has been recovered from the Starbon material. This indicates the repeatable runs of consecutive adsorption/desorption runs giving consistent results using different solvents.

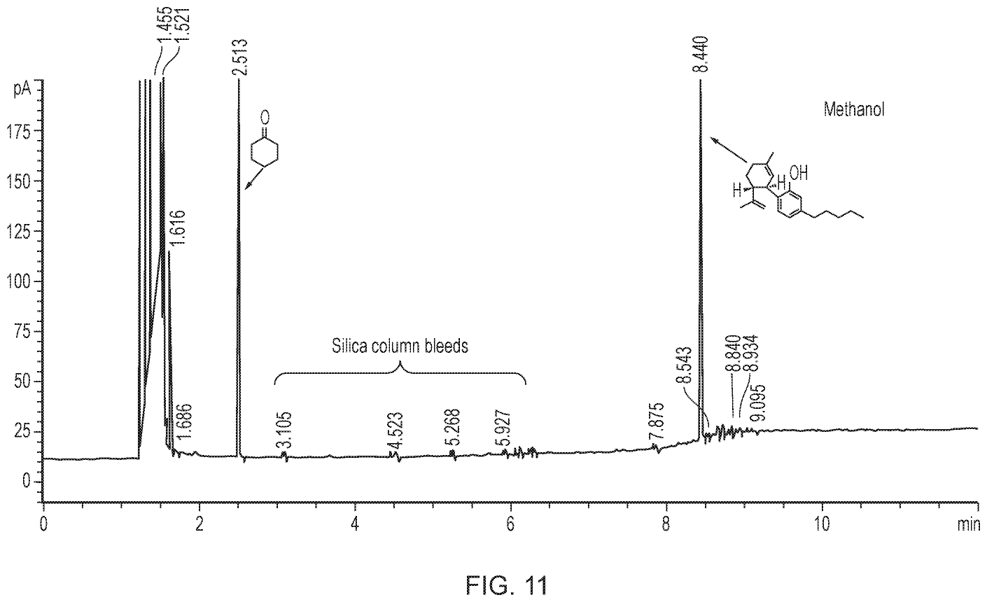

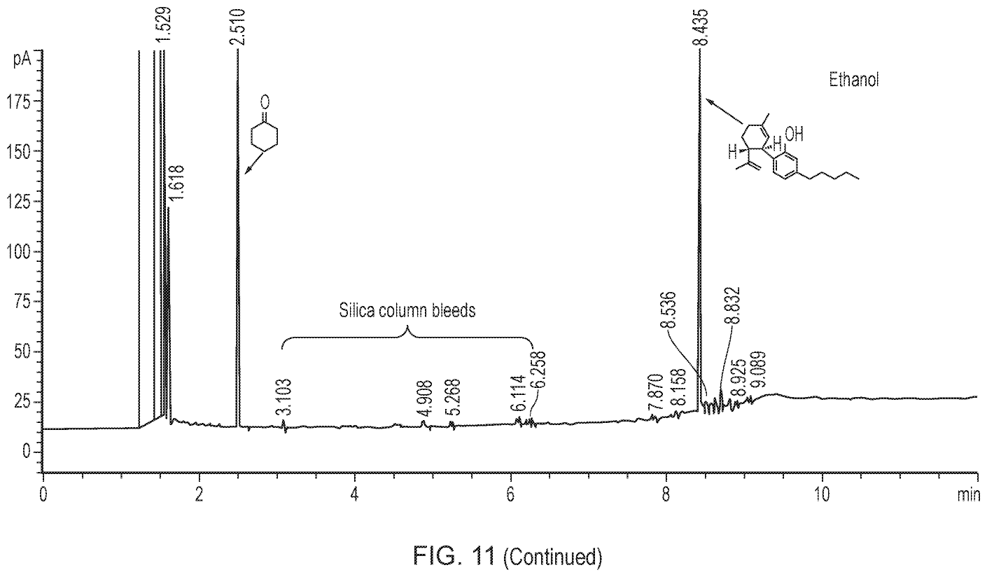

FIG. 11 shows GC chromatograms for desorption of HTs using hexane as the adsorption solvent and different desorption solvents (methanol and ethanol). The column bleed seen is due to the natural degradation of the silica stationary phase inside the column giving rise to a signal on the chromatogram. The levels of column bleed are below 5 pA*s and hence, seen as an acceptable. This aside, the desorption chromatograms can be seen to be free of non-cannabinoid compounds.

In addition to greening the desorption solvents, finding a replacement for hexane would be preferential as it is ranked as “hazardous” by the CHEM 21 solvent guide due to the following issues; hexane may be fatal if swallowed and enters airways, is toxic to aquatic life with long lasting effects, is a highly flammable liquid and vapour, is suspected of damaging fertility, may cause damage to organs through prolonged or repeated exposure, causes skin irritation and may cause drowsiness or dizziness. It is also a chemical of concern on the European Chemical Agency database. As there are not many non-polar, green solvents with sufficient volatility to allow recovery of extracts, supercritical carbon dioxide (scCO₂) was investigated as a replacement for hexane as the adsorption solvent. For these trials, A300 determined as the ideal adsorbent in month 1, was used to pack a column for adsorption/desorption of CBD using scCO₂ and ethanol as the solvent system.

In order to investigate this, a column packed with A300 was connected to a scCO₂ extractor vessel and a flow of CO₂ applied. This pre-conditioning at 150 bar, 50° C. and 30 g min⁻¹ CO₂ flow for 40 minutes removed any residual pyrolysis products from the solid A300 bed. Once complete, 300 mg of hemp extract, high in CBD (extracted with scCO₂ from hemp tops) and terpenoids, was loaded onto 8 g of Celite and placed into the extractor. Extraction was carried out at 350 bar 50° C., with 30 g min⁻¹ CO₂ flow for 2 hours. All extract removed from the celite during this time was passed through the bed of A300 before depressurisation. Compounds that did not adsorb onto the A300, precipitated out in the collector vessel and were removed with dichloromethane. Ethanol was then passed through the column to collect the compounds that adsorbed onto the A300.

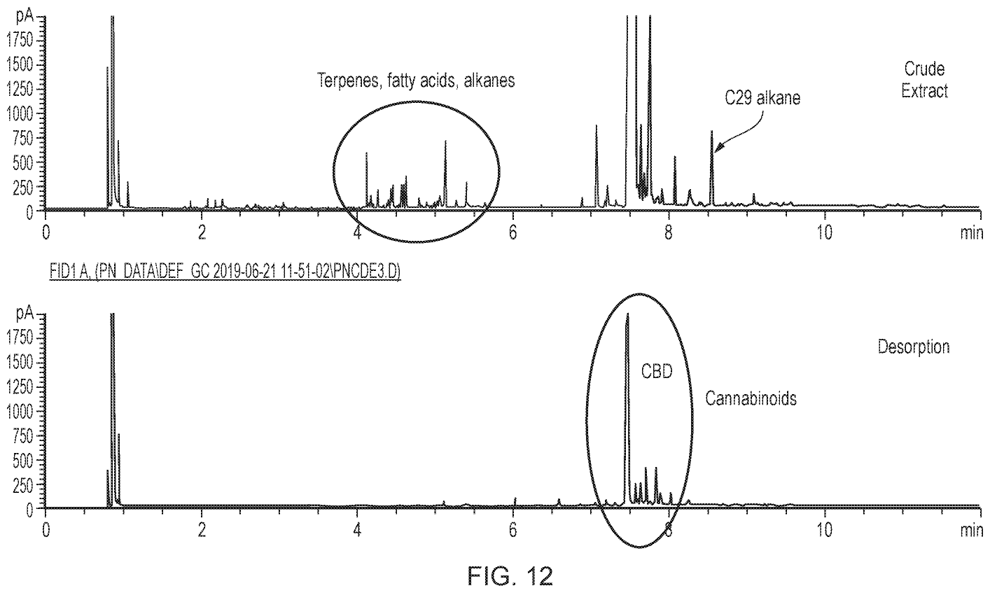

Post-extracts were analysed by means of GC-MS and GC-FID. Results are represented in FIG. 12, where it is clear to see that terpenes, hydrocarbons, some fatty acids passed straight through the A300 column and were removed with the scCO₂; i.e. no adsorption occurred indicating that as these compounds had no affinity towards the A300. Interestingly, the desorbed phase showed that the cannabinoids were preferentially adsorbed onto the A300, indicating that they could be separated using scCO₂ as the adsorption solvent. These results revealed that under scCO₂ conditions, terpenoids, hydrocarbons and waxes did not adsorb onto the A300 while the cannabinoids did.

Results indicated that selective isolation of high-value cannabinoids was achieved by utilising an A300 Starbon system coupled with scCO₂ adsorption and ethanol desorption. This novel system represents a key development in the extraction of high-value cannabinoids, as a one pot extraction/purification system represents an economic and scientific breakthrough compared to current high cost and environmentally detrimental techniques.

Adsorption and Desorption Capacity Testing—A-Series

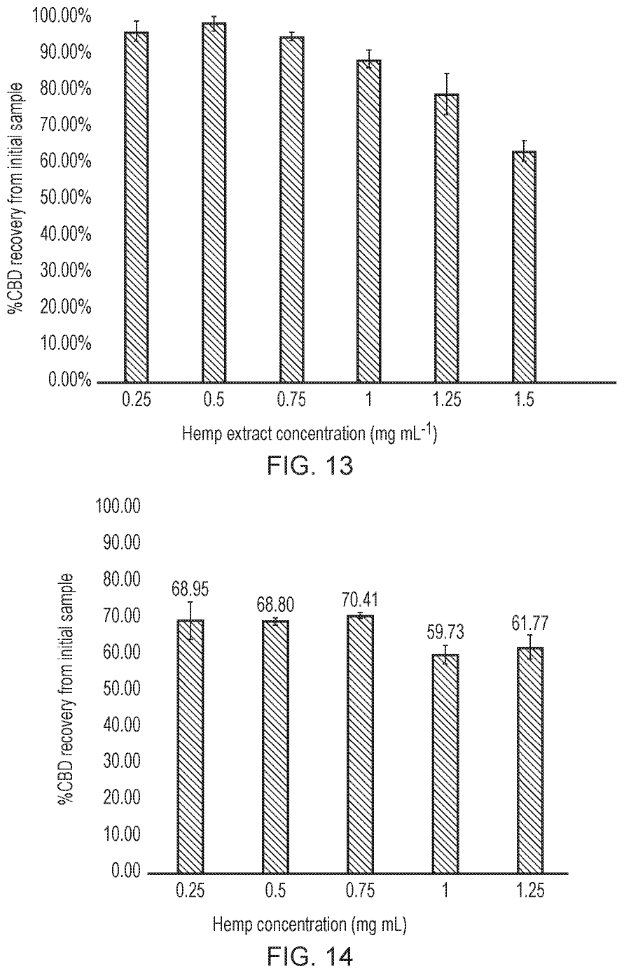

The adsorption capacity of A300 was investigated to see how much CBD could be loaded onto the A300 from the HT extract, thus determining the optimal loading of CBD. This was done by preparing a series of HT extract solutions of varying concentrations (mg extract mL solvent-1) and determining the percentage recovery of CBD for each concentration. The results are summarised in FIG. 13.

Low concentrations showed high levels of CBD recovery. All runs were repeated five times for reliability, which not only showed the ease of repeatability of this method but also the consistency of the A300 for multiple extraction runs. The level of CBD recovery seems to decrease nearer to 1 mg mL⁻¹ but is not deemed to be adverse until 1.5 mg/mL, with a recovery of 63%. Therefore, it was concluded that CBD recovery was adequate up to a concentration of 1.25 mg mL⁻¹. The level of cannabinoid and CBD recovery was also deemed to be better at the 1.25 mg mL⁻¹ compared to 1.0 mg mL⁻¹ (FIGS. 13 and 14).

Besides CBD recovery, adsorption capacity in terms of total cannabinoid recovery was also investigated. The total cannabinoid recovery was found to be greatest for 1.25 mg/mL over the 1 mg/mL but is lower for the 1.5 mg/mL. In fact, both CBD and total cannabinoid recovery results point to the 1.5 mg/mL being overloaded as it has the greatest material lost. Moreover, the level of adsorption is greater than desorption; therefore, more material passes directly through the Starbon as all the pores are full. The 1.25 mg/mL does lose more extraction material per run compared to the 1 mg/mL. As a result of this adsorption capacity study, it was concluded that the 1.25 mg/mL ensured the highest effective loading of the Starbon for not only CBD recovery but total cannabinoids also. However, it is important to note that these tests were done with a pre-set flow rate—the lowest possible flow rate that could be achieved with the multipurpose sampler. It is possible to get significantly better adsorption/desorption results by lowering the flow rate further—as demonstrated in the scale-up experiments.

The extractions were also investigated to see whether there was a difference between a Starbon that had been used for multiple extractions compared to a brand new Starbon cartridge. The hemp extraction was undertaken over 100 times throughout the adsorption capacity work. Adsorption capacity was shown to be effectively the same for both cartridges.

In fact, on a small scale, the cartridge septum broke before the adsorption capacity of the Starbon was seen to diminish.

Loading Capacity Testing—P Series

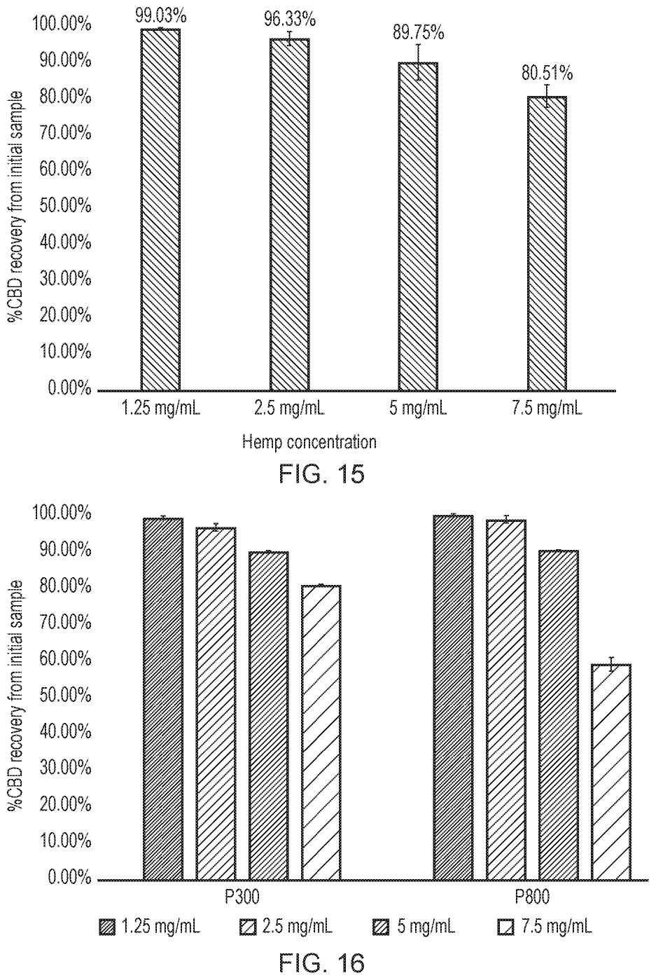

Similarly to the A-series, the P-series was then used to investigate the effect of increasing sequential loading of hemp extract on CBD recovery, in order to ascertain whether the adsorption capacity of the P-series is superior to the A-series. This was investigated due to the very high levels of recovery obtained at 1.25 mg/mL (FIG. 15). Interestingly, although CBD recovery decreased slightly with increasing hemp extract concentration, high levels of recovery were still observed. This could significantly accelerate the industrial use of the P-series solid-phase extraction material as it can extract CBD as efficiently as A300 material but at higher concentrations. The high level of recovery could be due to the P-series Starbons possessing a high mesopore to micropore ratio compared to A-series or S-series. IR-adsorption spectra of crude hemp extract and the scaled up Pecbons desorption work were compared and were consistent with the literature.

Recovery levels of CBD were tested at increased loading capacities across two Pecbons—P300 and P800 (FIG. 16). Recovery was seen to slightly decrease with increasing concentrations loaded onto the Starbon. However, it was seen that at certain increasing concentrations, there was decreased selectivity in the desorption of CBD, with small concentrations of fatty acids and other adsorption components coming through. The P300 and P800 both exhibit excellent recovery of CBD and, although both have similar recoveries, the P300 is slightly more selective in its desorption. Moreover, the adsorption of CBD onto the P800 is better than the P300. This could be due to the increased microporosity exhibited by pecbons with increasing preparation temperature. However, the Starbon prepared at lower temperatures is more industrially viable as it is cheaper to produce as similarly to the A300 less expanded material is lost during pyrolysis. Therefore, the P300 was chosen for the scaled-up extractions.

Scale Up of Starbon Algibon A300

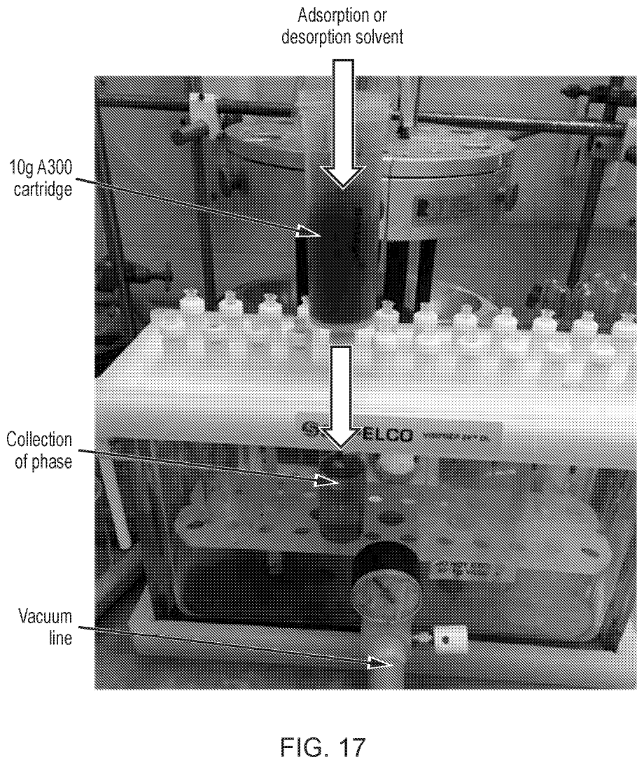

The A300 Starbon was scaled up from an 18 mg scale to a 10 g scale to assess the potential industrial viability (FIG. 17). This would represent a potential step forward for green extraction processes. The same method and washing steps were carried out manually in line with the MPS sequence. The extraction was run twice to negate the effect of the first extraction being inconsistent, as explained previously. As with the small-scale work, conditioning of the Starbon was vital as the first extraction run gave a poor CBD recovery in the desorption phase. A CBD recovery of 97% was extracted on the second run and quantified via the external standard. This is compared with a value of 82% obtained on the ITSP cartridge.

The flow rate of the large-scale experiment was slower than the 10 uLs⁻¹ of the small-scale work due to the maximum vacuum that could be applied. This is a potential reason for the higher extraction yield of CBD observed. The ratio of extract to Starbon was kept the same as MPS extraction experiments and the volume of solvents used for the adsorption/desorption was scaled appropriately. A 0.3 mL sample at 1.25 mg ml⁻¹ isolated on an 18 mg cartridge gives a loading of 20.8 mg of extract per g of A300. Higher concentrations and smaller volumes of solvent may give similar or potentially better results. Contact time between the extract and the Starbon is greater here than in the small-scale automated work suggesting the system requires time to fully equilibrate.

Hydrogen Nuclear Magnetic Resonance (¹H NMR) of Scaled Up Algibons

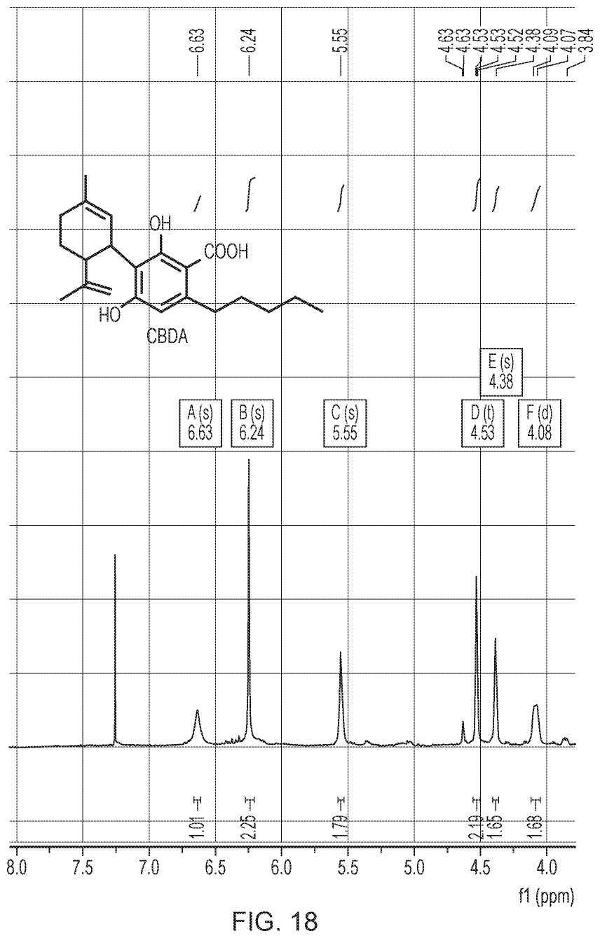

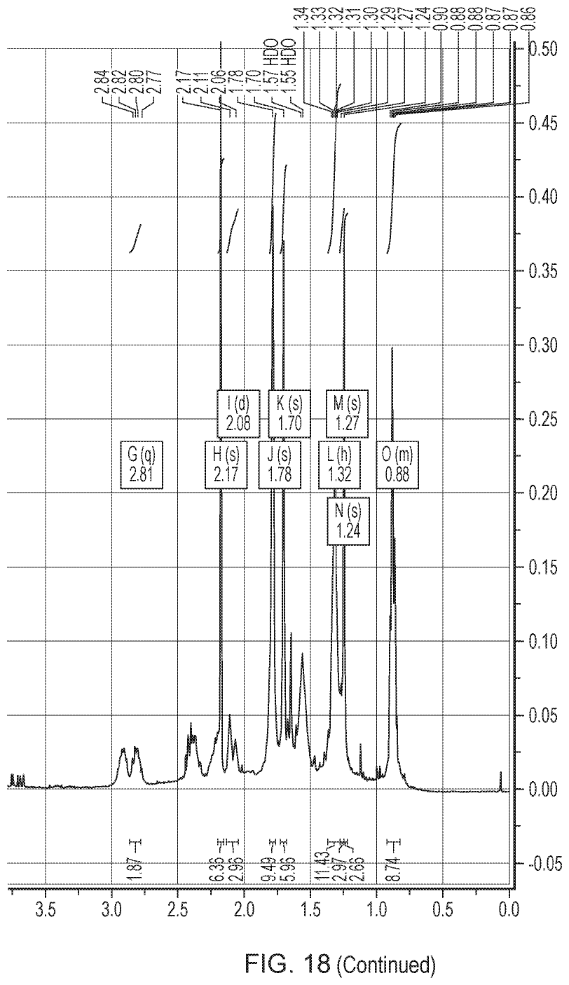

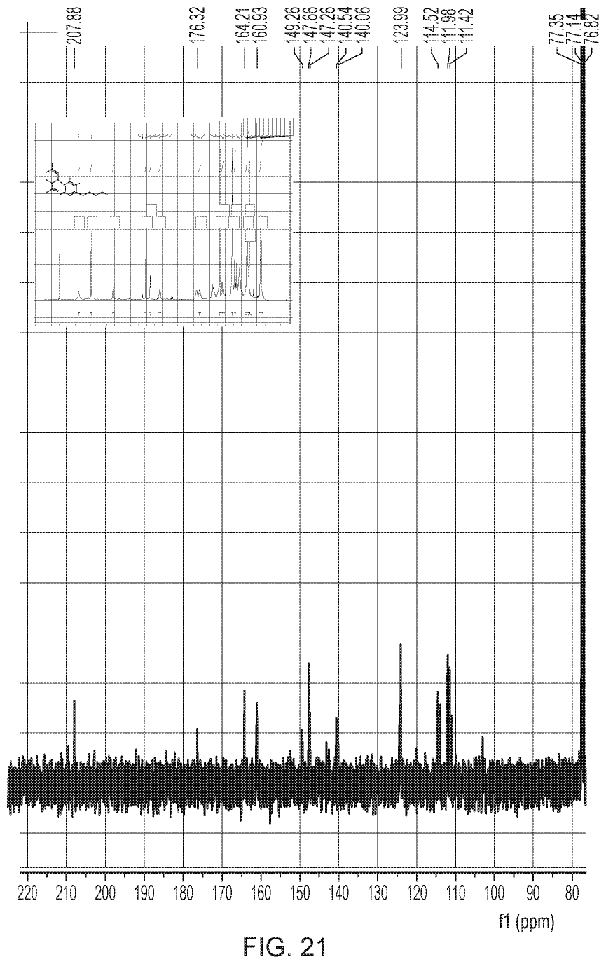

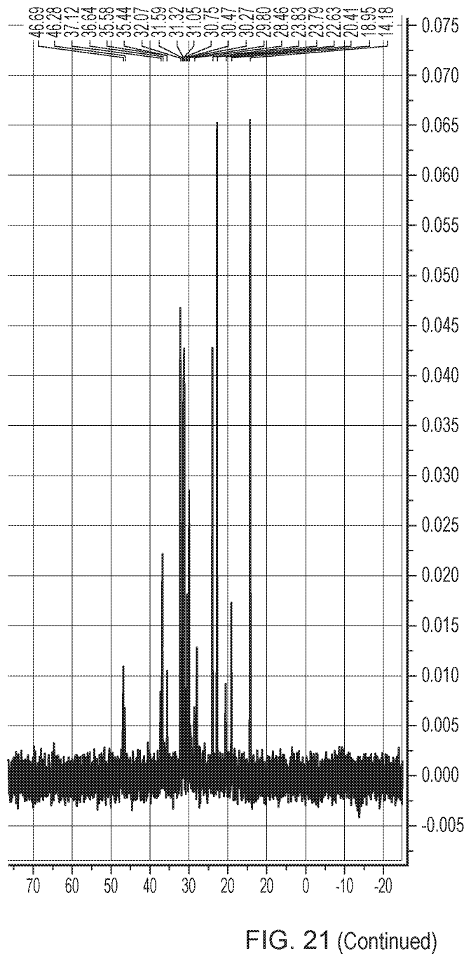

¹H NMR ran of the large scale of Algibons (A300) showed that it was in fact CBDA that was isolated. This was confirmed by 1H NMR (FIG. 18) as well as ¹³C NMR (FIG. 21).

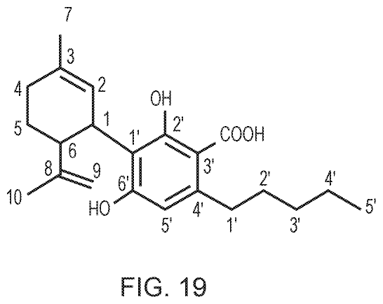

Key indicators that show the isolated product is CBDA is the peak at 175.6 ppm in the 130 NMR for a COOH group. Additionally, signals at C-1′, C-2′, C-4′, C-5′, C-6′ shift downfield in ¹³C NMR in CBDA compared to CBD due to the hydrogen bonding of the hydroxyl and carboxylic groups. Full characterisation of both ¹H NMR and ¹³C NMR was undertaken and was comparable to what has been previously observed in the literature. Labelling and assignment of NMR peaks was in accordance with prior literature as illustrated in FIG. 19. The area of the 1H NMR spectra that gives the most distinguishing information about the cannabinoids is in the 1H NM 4-6 ppm.

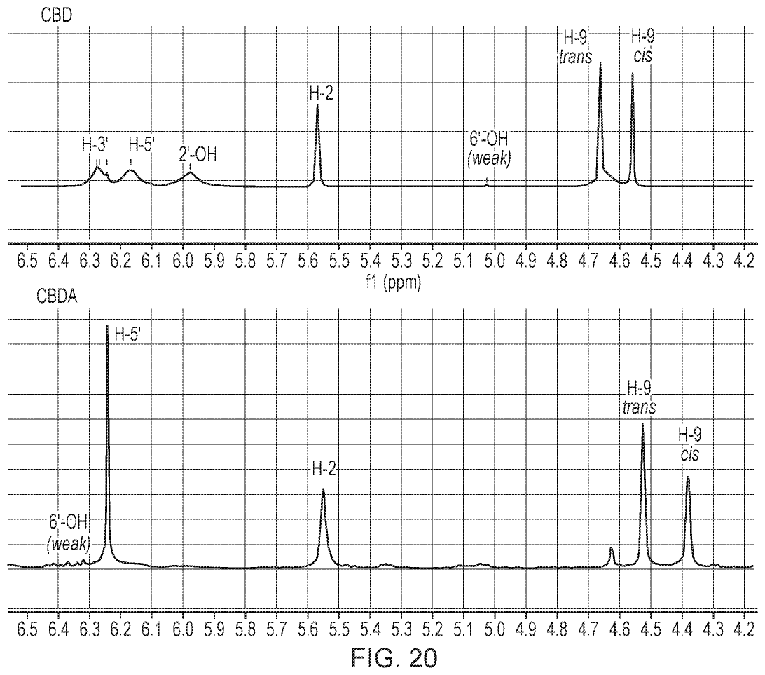

Furthermore, in the 1H NMR of spectrum of CBD measured in CDCl₃, broad peaks were observed that were unchanged with the addition of deuterium oxide, thus indicating they are aromatic hydrogens and not hydroxyl moieties (FIG. 20).

Scale Up of Starbon Pecbon P300

A scale up of the Pecbon was run using the same parameters as the MPS using a 3 g Pecbon cartridge and the same washing and extraction steps used for the Algibon large-scale work. The large-scale extraction gave a 99% CBD recovery.

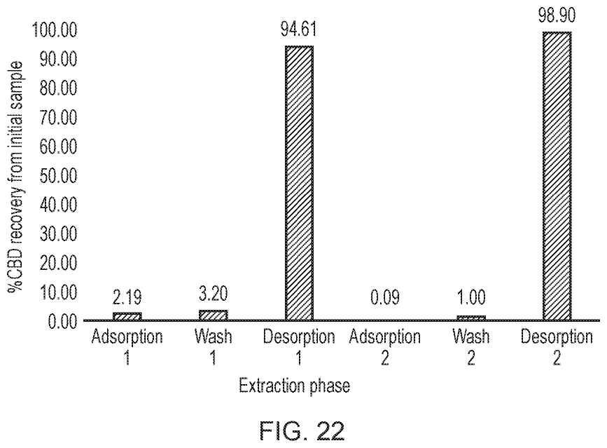

The amount of CBD recovered from desorption of Pecbons was comparable to large-scale Algibons. The washing steps implemented during method optimisation on the MPS scale work were used to show that little CBD is left on the Starbon after each adsorption/desorption run. Moreover, the Starbon was weighed prior to any extractions, as well as after, and showed an identical mass. This means that washing steps and extractions give no large masses of residual bio-oils or hemp components stuck to the cartridge that could affect sequential extraction runs. This not only enhances the reproducibility of extraction results but allows for reusability of a large-scale Starbon cartridge. FIG. 22 also shows the reusability of the Starbon as large-scale extractions show minimal CBD is lost between runs, and the majority of the CBD eluted from the Starbon in the designated desorption steps (<90% CBD desorption per run). Again, in order to carry out a like for like consideration, loading of HT extract was kept at 1.25 mg mL⁻¹, although experiments in the scale up of A300 and P300 indicates that a 6-fold increase to 7.5 mg mL⁻¹ would give similar results. A 0.3 mL sample at 7.5 mg ml⁻¹ isolated on an 18 mg cartridge gives a loading of 124.8 mg of extract per g of P300.

One Pot System

In this case, hemp dust was utilised as feedstock as the number and complexity of additional compounds in the extract was significantly higher than that found in hemp tops including long-chain hydrocarbons, saturated and unsaturated fatty acids, fatty alcohols, fatty aldehydes, wax esters and sterols. Additionally, the cannabinoid content in this extract is lower. This system was trialed at both 350 bar, 50° C. and 400 bar, 60° C., with the latter representing the optimised extraction conditions for highest yield from the hemp dust tested. Results were close enough to be considered identical under both conditions investigated.

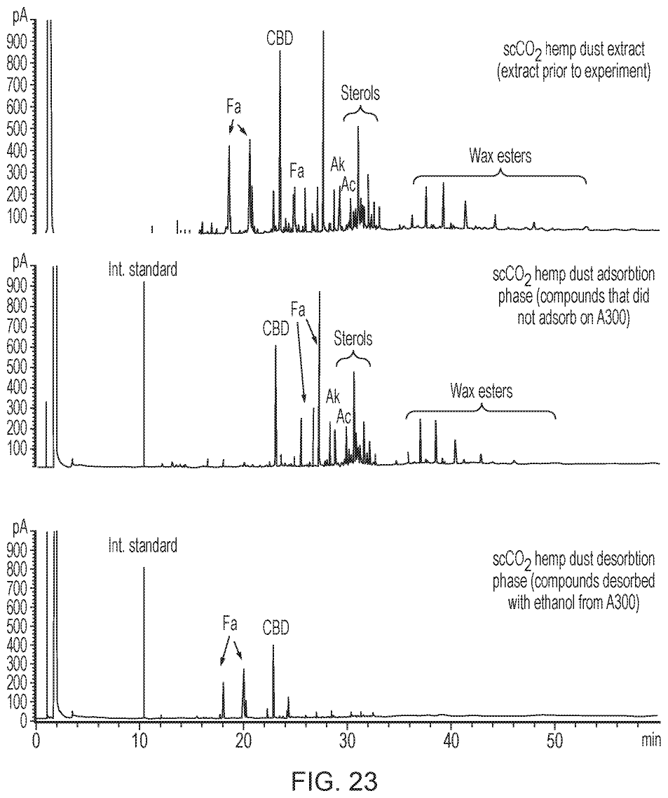

Separation of cannabinoids from the crude extract was confirmed by means of GC-MS data. Two types of extracts were analysed. FIG. 23 represents recovered fractions:
1. An extract containing compounds that did not adsorb on to the A300 (passed directly through the starbon column and collected in the separator). This is analogous to the adsorb solvent phase in hexane.
2. An extract containing compounds that were strongly bound onto the A300, and subsequently desorbed in ethanol.

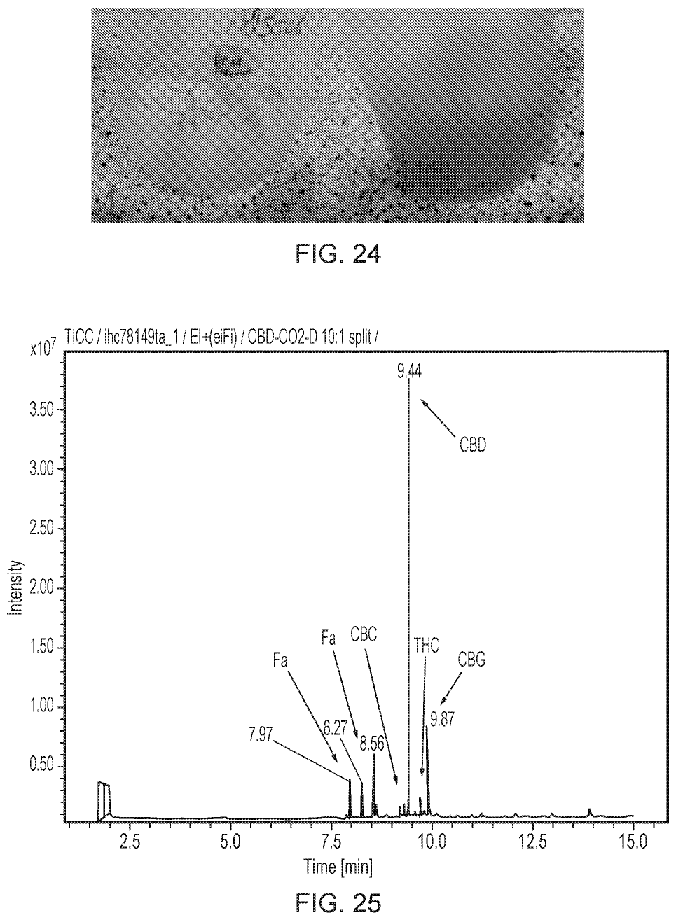

The visual difference between the two extracts (texture and colour) is clearly seen in FIG. 24. The material that did not adsorb onto the starbon is clearly solid and waxy in character while the material that desorbed in ethanol is oily.

Both FIGS. 23 and 24 confirm that the majority of compounds within the hemp dust extract have little to no affinity for the starbon solid phase. The cannabinoids, however, bind sufficiently strongly to be retained by the starbon even under high temperature and pressure. FIG. 23 shows cannabinoids present in the adsorption fraction, most likely due to the overloading of the mesopores within A300 (60 g of hemp dust relates to 0.66 g of extract, separated using 8 g of starbon). Potentially under the high-pressure conditions some cannabinoids may be removed by scCO₂ and/or the supercritical fluid is competing with the organic compounds to bind to the Starbon.

GC-EI-MS analysis conducted on the one pot hemp dust desorbed phase showed the following cannabinoids; Cannabidiol (CBD), Cannabigerol (CBG), Cannabichromene (CBC) and Tetrahydrocannabinol (THC) (FIG. 25). Interestingly CBG, CBC and THC were not previously observed in scCO₂ hemp dust extracts carried out without solid phase extraction (SPE). This confirms that Starbon SPE enriches the cannabinoid content as compared to the crude extract. All cannabinoids appear to have affinity to the Starbon material, although some may do so more preferentially.

### CONCLUSION

The isolation and purification of cannabinoids from HT extract was successfully conducted using various renewable mesoporous carbonaceous materials, Starbons. The A300 showed the greatest recovery of CBD (60%) amongst the A-series at 1.00 mg mL⁻¹ HT extract (20 mg of hemp extract per g of Starbon). This work also trialed novel adsorption/desorption work on pectin based Starbons, furthering the uses of renewable mesoporous materials as a sustainable means of natural product separation. P300 and P800 both showed outstanding recoveries of CBD (>99%) from HT extracts.

It has been demonstrated that replacing methanol with ethanol as the desorption solvent increased the yield of CBD desorbed (83.97±3.5%) whilst also incorporating a solvent into the process that is not only greener, but also a food-grade solvent. The data indicates that replacement of hexane as the adsorption solvent with scCO₂ is possible, with cannabinoids selectively adsorbed onto the A300 and therefore isolated from other constituents of the extract.

Adsorption capacity work on A300 and P300 showed appreciable differences between various loadings on HT extract. The optimum concentration for the A300 was determined, via CBD yield and CBD purity, to be 1.25 mg mL⁻¹ and was subsequently used in further work. The Pecbons showed greater loading capacities compared to A-series. The largest amount of HTs, loaded at 7.5 mg mL⁻¹, still exhibited an 80.15±0.03% CBD recovery.

Large-scale experiments of A300 (10 g) and P300 (3 g) material were used for a scaled-up adsorption/desorption run of HTs that showed a CBD recovery of >97%. A slower flow rate, compared to the autosampler, could be a reason for the higher level of CBD recovery. Large-scale desorption product 1H NMR analysis also showed the acidified form of CBD, CBDA, is separated from HTs.

Finally, the coupling of scCO₂ extraction with the Starbon purification technology was attempted in order to investigate whether it is possible to combine extraction and purification of cannabinoids in a one-pot system. Results demonstrated that the majority of compounds found within the hemp dust extract have little to no affinity for the starbon solid phase. The cannabinoids, however, bind sufficiently strongly to be retained by the starbon even under high temperature and pressure). Interestingly CBG, CBC and THC, which were not previously observed in scCO₂ hemp dust extracts carried out without solid phase extraction (SPE), were detected in the extracts with SPE. This confirms that Starbon SPE enriches the cannabinoid content as compared to the crude extract. All cannabinoids appear to have affinity to the Starbon material, although some may do so more preferentially.

1. A method of isolating one or more cannabinoids from a natural product by solid phase extraction, wherein the method comprises:
(a) contacting the natural product with a primary solvent to obtain a feed solution;
(b) passing the feed solution through a mesoporous material, thereby adsorbing the one or more cannabinoids to the mesoporous material;
(c) passing a wash solution through the mesoporous material; and
(d) passing a second solvent through the mesoporous material, thereby desorbing the one or more cannabinoids from the mesoporous material into an elution solution, thereby isolating the one or more cannabinoids.

2. The method of claim 1, wherein:
(i) the natural product is non-extracted plant material; or
(ii) the natural product is extracted plant material,
optionally wherein the natural product is derived from Cannabis.

3. (canceled)

4. (canceled)

5. The method of claim 1, wherein the one or more cannabinoids comprise cannabidiol (CBD) and/or cannabidiolic acid (CBDA).

6. The method of claim 1, wherein the primary solvent is non-polar, optionally wherein the non-polar solvent is hexane or supercritical CO₂.

7. (canceled)

8. The method of claim 1, wherein the second solvent is polar, optionally wherein the polar solvent is ethanol.

9. (canceled)

10. The method of claim 1, wherein the wash solution comprises the primary solvent.

11. The method of claim 1, wherein the mesoporous material is functionalised with aromatic and/or hydroxyl groups.

12. The method of claim 1, wherein the mesoporous material comprises:
(i) mesopores having an average diameter between about 2 to about 50 nm;
(ii) mesopore volumes (Vₘₑₛₒ) between about 0.2 to about 2.0 cm³ g⁻¹;
(iii) a surface area of at least about 150 m² g⁻¹ or more; and/or
(iv) pores in which a ratio of the mesoporous volume (Vₘₑₛₒ) to a microporous volume (V_{micro}) is greater than about 10.

13. The method of claim 1, wherein the mesoporous material is polysaccharide-derived mesoporous carbon, wherein optionally:
(i) the mesoporous material is derived from a polysaccharide having acid-functionality; or
(ii) the mesoporous material is derived from alginic acid or pectin, optionally wherein the mesoporous material has been obtained by a thermal treatment regime between about 300° C. to about 800° C.

14. (canceled)

15. (canceled)

16. The method of claim 1, wherein:
(i) a pre-treatment step of passing a wash solution through the mesoporous material is performed prior to passing the feed solution through the mesoporous material; and/or
(ii) the content of one or more cannabinoids is determined in the feed and/or wash solution after passing through the mesoporous material, optionally wherein the feed and/or wash solution is re-passed through the mesoporous material.

17. The method of claim 1, further comprising:
(e) removing any second solvent that may be retained on the mesoporous material after the second solvent has been passed through the mesoporous material, optionally by air or vacuum drying; and/or
(f) reconditioning the mesoporous material for repeated use after the second solvent has been passed through the mesoporous material, optionally by passing the wash solution through the mesoporous material.

18. The method of claim 1, wherein:
(i) the feed solution comprises a concentration of about 1.25 mg/mL or more of extracted plant material; or
(ii) the feed solution, wash solution and/or second solvent is passed through the mesoporous material at a flow rate allowing contact with the mesoporous material for between about 1 to about 5 minutes.

19. (canceled)

20. The method of claim 1, wherein the mesoporous material is housed within a column, optionally wherein a column volume of feed solution, wash solution and/or second solvent is passed through the mesoporous material.

21. (canceled)

22. The method of claim 1, wherein the second solvent is removed from the elution solution.

23. The method of claim 1, wherein the elution solution is a cannabinoid-rich extract.

24. A method of making a pharmaceutical composition comprising one or more cannabinoids as an active ingredient, wherein the method comprises:
(i) isolating one or more cannabinoids according to the method of claim 1;
(ii) optionally separating CBD and/or CBDA from one or more other cannabinoids in the elution solution; and
(iii) formulating the isolated cannabinoid(s) with one or more pharmaceutically acceptable diluents, carriers or excipients.

25. Use of a mesoporous material as defined in claim 11 in isolating one or more cannabinoids from a natural product.

26. A cannabinoid-rich extract obtained by the method of claim 1.

27. The cannabinoid-rich extract obtained by the method of claim 26, wherein the cannabinoid is CBD and/or CBDA, optionally wherein the cannabinoid-rich extract comprises detectable levels of Cannabigerol (CBG), Cannabichromene (CBC) and Tetrahydrocannabinol (THC) as measured by gas-chromatography-electron impact-mass spectrometry (GC-EI-MS) analysis.

28. (canceled)

29. The cannabinoid-rich extract of claim 26, wherein the extract has the GC-EI-MS profile substantially as shown in FIG. 25.
